(19) United States
(12) Patent Application Publication
(10) Pub. No.: US 2021/0267438 A1
(43) Pub. Date: Sep. 2, 2021

(54) MINIATURIZED INTRA-BODY CONTROLLABLE MEDICAL DEVICE EMPLOYING MACHINE LEARNING AND ARTIFICIAL INTELLIGENCE

(71) Applicant: Miraki Innovation Think Tank, LLC, Cambridge, MA (US)

(72) Inventors: Christopher J. Velis, Cambridge, MA (US); Matthew P. Palmer, Medford, MA (US); Nisha Veronica Varma, Brookline, MA (US)

(21) Appl. No.: 17/254,399

(22) PCT Filed: Jun. 24, 2019
(86) PCT No.: PCT/US19/38695

§ 371 Date: Dec. 21, 2020

Related U.S. Application Data
(60) Provisional application No. 62/691,859, filed on Jun. 29, 2018.

(51) Int. Cl. A61B 1/00; A61B 10/04; A61B 10/00; A61B 5/15

(52) U.S. Cl. CPC A61B 1/00156; A61B 1/00105; A61B 1/00016; A61B 1/00006; A61N 5/062; A61B 10/04; A61B 10/007; A61B 10/0038; A61B 5/150206; A61B 1/00032

(57) ABSTRACT

A medical system includes one or more medical devices for intra-body conveyance which include a host structure that defines an interior area, one or more data gathering system, one or more means for communication, for transmitting data from the data gathering systems. The medical device is configurable into a peripheral boundary of a size adapted to fit in a lumen of a living organism. The medical system includes an external processing device configured to receive the transmitted data from the means for communication. The external processing system is configured to perform data analysis on the data received from the medical device.

### FIELD OF THE INVENTION

The present invention relates generally to a miniaturized intra-body controllable medical device. More specifically, the invention relates to the intra-body medical device utilizing machine learning and artificial intelligence (herein referred to as AI) to control and guide the actions of the device. The device may, in real time, use algorithms to analyze data obtained from the imaging and sample and data gathering system to identify potential patient pathologies. The AI may compare the patient's data to a database of clinical data, while taking into account the patient's own medical history, assess the findings, make a diagnosis, and/or provide treatment.

### BACKGROUND OF THE INVENTION

Many medical procedures require a physician to gain access to regions within the body in order to complete a diagnosis or provide therapy to a patient. Often, physicians access internal regions of the body through the body's own natural orifices and lumens. Natural orifices include the nostrils, mouth, ear canals, nasolacrimal ducts, anus, urinary meatus, vagina, and nipples. The lumens include the interior of the gastrointestinal tract, the pathways of the bronchi in the lungs, the interior of the renal tubules and urinary collecting ducts, the pathways of the vagina, uterus, and fallopian tubes. From within these orifices and lumens, physicians can create an incision to gain access to almost any region of the body.

Traditional methods for gaining access to regions within the body include open surgical procedures, laparoscopic procedures and endoscopic procedures. Laparoscopic procedures allow the physician to use a small “key-hole” surgical opening and specially designed instruments to gain access to regions within the body. Initially, laparoscopic instruments were linear in nature, and required a straight obstruction free “line-of-sight” to access regions of the body. Endoscopic procedures allow the physician to access regions of the digestive system by passing flexible instruments through either the mouth or rectum.

Recently, physicians have begun to control these instruments using robots. These robots are typically connected in master/slave configuration, where the robot translates the physician's movements into instrument movements. Robotic controls have also allowed for advent of flexible laparoscopic instruments. Medical robots still require a physician to be actively controlling the movements and actions of the devices being controlled and require large expensive capital equipment and dedicated operating room spaces.

Additionally, pill capsules have been invented that allow for a patient to ingest the capsule and as it passes through the digestive system takes pictures. There are no means for: controlling the motion of these capsule devices, tracking or controlling the orientation, speed or location of these capsule devices, accurately knowing where pictures were taken by the capsule devices, and performing any type of surgical procedure or delivering therapy with the capsule devices.

Thus, improvements are desirable in this field of technology. It would be beneficial to combine the ability to perform surgical procedures and provide therapy using robotic instruments with the footprint, size, and maneuverability of capsule systems or other structures. It would be beneficial to provide a means for controlling the movement of a medical device so that the surgeon can navigate it to a specific location.

Additionally, tissue samples and medical images from a patient are commonly used to make a clinical diagnosis. Medical imaging enables visual representations of the interior of a body for clinical analysis and medical intervention. Over the last two decades medical imaging has undergone profound changes and the perspective has changed from diagnosing evident disease to the detection of subtle abnormalities. Newer imaging techniques attempt to visualize what was formerly possible only with biopsy and histologic interpretation. With the advent of high resolution, high definition medical imaging, the problem of sifting through voluminous medical data taken across large regions of the body will become increasingly overbearing to physicians. The problem becomes even more profound when the medical data is multi-variate including not only high resolution and high definition images but other clinically relevant data like temperature, pressure, and pH level. Thus, there needs to be development of AI algorithms to automate the analysis of medical images, tissue samples and other clinically relevant data.

### SUMMARY

There is disclosed herein a medical device for intra-body conveyance that includes a host structure that defines an interior area. The medical device includes one or more propulsion systems that are linked to the host structure. The host structure and the propulsion systems are configurable into a peripheral boundary of a size adapted to fit in a lumen of a living organism. The medical device includes one or more power supplies that are in communication with the one or more of the propulsion systems. The medical device includes a control unit that is in communication with one or more or the propulsion systems and the power supplies. The control unit has a computer process controller that is configured to control the propulsion systems to move the host structure and the propulsion systems in the lumen so that the host structure and the propulsion systems are self-maneuverable within the lumen.

The propulsion systems may include a sprocket driven track structure in communication with the host structure; a fluid jet stream discharging from the host structure; a plurality of articulating tentacles extending from the host structure; a screw-drive formed on external surfaces of the host structure; a pull device and/or a push device in communication with the host structure; and an arrangement of inflating and deflating balloons, the balloons being in predetermined positions on the host structure, and/or in predetermined positions around the host structure.

In one embodiment, the propulsion systems include an orientation control device configured for orientation control of the medical device within the lumen. The orientation control devices and the propulsion systems include, for example, stabilization wings, flippers, anchors, braces, supports, clamps, a gyroscope and/or a ballast systems.

In one embodiment, the medical device includes a docking station for receiving a tether, a medical scope and/or a second medical device. In one embodiment, the medical scope is ENT otoscope, a naso-pharyngoscope, a laparoscope, a sinuscope, a coloposcope, a resectoscope or a cystoscope. In one embodiment, the docking station includes the tether, a holding device, a release device, a launch device, a push device and/or a pull device.

In one embodiment, the medical device 5, includes a tracking device, a signal transmitter and a signal receiver in communication with the control unit for tracking and guiding the medical device within the lumen.

The power supplies include miniaturized batteries, fuel cells, electrochemical reactors, piezoelectric devices, energy harvesting devices that obtains thermal and/or chemical reaction energy from the fluids in and tissue of the lumen and adjacent organs, thermal reactors, heat absorption energy conversion devices and triboelectric energy harvesting devices.

In one embodiment, the host structure includes a storage system that has miniaturized compartments for housing one or more power supplies, energy storage devices, medications, imaging systems, computer processor controllers, communications transmitters and receivers, propulsion systems, therapy delivering devices (e.g., radiation sources), process waste, biopsies, blood and tissue samples, medical and surgical instruments, fluids, gases, powders and consumables.

In one embodiment, the host structure includes a clinically inert material, a sterilizable material, an elastomeric material, a chemically reactive material, a chemically inert material, a disintegrable material, a dissolvable material, a collapsible material and a material having physical and chemical properties to withstand exposure to bodily fluids for predetermined periods of time.

In one embodiment, the host structure includes an imaging system such as X-ray radiography, magnetic resonance imaging, medical ultrasonography or ultrasound, confocal microscopy, elastography, optical-coherence tomography, tactile imaging, thermography and medical digital photography. In one embodiment, the imaging system is configured to travel through the lumen in the medical device. In one embodiment, one or more of the imaging systems is configured to be discharged from the medical device while in the lumen and deposited in a predetermined location in the lumen for ongoing monitoring.

In one embodiment, the host structure includes one or more therapy delivery systems such as optical-coherence tomography (OCT) guided laser instruments, radiation discharging sources, chemotherapy deploying devices, pharmaceutical and drug deploying devices, and photodynamic therapy devices. In one embodiment, one or more of the therapy delivery systems is configured to travel through the lumen in the medical device to provide therapy. In one embodiment, the one or more of the therapy delivery systems is configured to be discharged from the medical device while in the lumen and deposited in a predetermined location in the lumen for ongoing therapy delivery. In one embodiment, one or more of the therapy delivery systems is configured with a storage medium configured to record time, duration and/or application location of the therapy.

In one embodiment, the host structure includes a sample gathering system and/or a data gathering system.

In one embodiment, the sample gathering system is configured to obtain tissue biopsies and blood, bone, cells, bone marrow, blood, urine, DNA and/or fecal samples.

In one embodiment, the data gathering devices include one or more pH probes, accelerometers, pressure transducers, thermometers, and/or dimensional measurement systems.

In one embodiment, the host structure includes one or more material dispensing systems equipped with storage compartments configured for storing and/or dispensing payloads such as medication, liquids, powders, chemically reactive agents and radiation emitting sources.

In one embodiment, the material dispensing system includes an actuator, a pump, a compressor, a nozzle, a flow control device, an injection device, a piercing device a dose measuring device and a recording device.

In one embodiment, the present invention includes an interactive group of at least two of the medical devices, wherein the interactive group of the medical devices are in communication with an external computer-based control system and/or each other and are configured to cooperate with each another to perform predetermined missions or tasks.

The present invention includes a method for using the medical device administering medications, administering therapy, deploying medical devices, imaging and/or surgery.

The present invention includes a method of using the medical device for use in a gastro/intestinal tract, use in urology applications, use in a lung, use in a bladder, use in a nasal system, use in a reproductive system, use in performing Transurethral Resection of Bladder Tumors (TURBT), use in Transurethral Resection of the Prostate (TURP), use in trans rectal prostate ultrasound, biopsy, and radiation treatment.

The present invention includes employing the medical devices for use in procedural environments, operatory and surgical procedures, ambulatory and out-patient procedures and/or unobtrusive normal routine living.

In one embodiment, a plurality of the medical devices are in communication with one or more repositories such as a heat sink, a chemical reactor and a storage vessel. In one embodiment, the plurality of medical devices includes a cooling system and a material discharge system, wherein the repository is positioned intra body or outside the body.

The present invention includes a medical system that employs one or more of the a medical devices and is configured to perform data analysis on the data received from the medical device, wherein the data analysis includes machine learning and artificial intelligence.

There is disclosed herein a medical system that employs one or more of the medical devices for intra-body conveyance. The medical devices include a host structure that defines an interior area and one or more data gathering systems. The medical device includes one or more means for communication for transmitting data from the data gathering systems. The medical device is configurable into a peripheral boundary of a size adapted to fit in a lumen of a living organism. The medical system includes a processing device (e.g., external to the body or internal to the body or medical device) configured to receive the transmitted data from the means for communication and is configured to perform data analysis on the data received from the medical device.

In one embodiment, the data from the data gathering system is a group consisting of images, pH values, temperatures, positions, forces, pressures, dimensions, time, and combinations of the foregoing.

In one embodiment, the images are obtained by white light, contrast enhancement using dye, optical methods, electronic methods, narrow band imaging, auto fluorescence, confocal laser microscopy, optical coherence tomography, fluorescence, reflectance spectroscopy, targeted imaging, and multimodal imaging.

In one embodiment, the external processing device is s a local computer terminal, a cloud computer terminal or a portable device.

In one embodiment, the data analysis is performed by the external processing device and includes a review of the data for unusual patterns and anomalies and can further include application of instructions and algorithms to the data to compare data received from the medical device to data stored in a database in communication with the external processing device.

In one embodiment, the review of the data for unusual patterns and anomalies includes analysis of gross shape; morphology; cell shape; size; nuclei shape; size and number of nuclei; structure of chromatin; scattering properties; pH level, temperature level; pressure; and tactile level.

In one embodiment, the data analysis provides a probability of abnormality including those of neoplastic lesions, ulcers and polyps.

In one embodiment, the data analysis provides recommendations for next steps such as medical related next steps to be performed by the medical device.

There is disclosed herein a method of diagnosing or treating one or more anomalies in a patient. The method includes placing one or more of the medical devices into a lumen or an orifice of a patient, collecting data about the patient with the medical device, transmitting the data about the patient from the medical device to an external processing device, applying instructions to the data received from the medical device to analyze the data; and utilizing the data analysis to diagnosis or treat the patient.

In one embodiment, the data includes images, pH values, temperatures, positions, forces, pressures, dimensions, time, and combinations of the foregoing.

In one embodiment, the images are obtained by white light, contrast enhancement using dye, optical methods, electronic methods, narrow band imaging, auto fluorescence, confocal laser microscopy, optical coherence tomography, fluorescence, reflectance spectroscopy, targeted imaging, and/or multimodal imaging.

The external processing devices include a local computer terminal, a cloud computer terminal, and a portable device.

In one embodiment, the data analysis is performed by the external processing device and includes a review of the data for unusual patterns and anomalies, and application of instructions or algorithms to the data, to compare data received from the medical device to data stored in a database in communication with the external processing device.

In one embodiment, the review of the data for unusual patterns and anomalies includes analysis gross shape; morphology; cell shape; size; nuclei shape; size and number of nuclei; structure of chromatin; scattering properties; pH level, temperature level; pressure; and tactile level.

In one embodiment, the data analysis provides a probability of abnormality including those of neoplastic lesions, ulcers and polyps.

The method may include a data analysis which provides recommendations for next steps, including medical related next steps performed by the medical device.

### DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A illustrates a representative intra-body controllable medical device formed in accordance with the present invention.

FIG. 1B illustrates a representative intra-body controllable medical device formed in accordance with the present invention.

FIG. 2 illustrates an alternative representation of an intra-body controllable medical device formed in accordance with the present invention.

FIG. 3 illustrates an intra-body controllable medical device featuring a helical screw drive propulsion system formed in accordance with the present invention.

FIG. 4 illustrates an intra-body controllable medical device featuring a sprocket driven track propulsion system formed in accordance with the present invention.

FIG. 5 illustrates an alternative representation of an intra-body controllable medical device featuring a sprocket driven track propulsion system formed in accordance with the present invention.

FIG. 6 illustrates an intra-body controllable medical device featuring a fluid/jet stream propulsion system formed in accordance with the present invention.

FIG. 7 illustrates an intra-body controllable medical device featuring a tentacle propulsion system formed in accordance with the present invention.

FIG. 8 illustrates an alternative representation of an intra-body controllable medical device featuring a tentacle propulsion system formed in accordance with the present invention.

FIGS. 9A, 9B and 9C illustrate an intra-body controllable medical device featuring an anchor and tether propulsion system formed in accordance with the present invention.

FIGS. 9D and 9E illustrate an intra-body controllable medical device featuring a push type propulsion system formed in accordance with the present invention.

FIGS. 9F and 9G illustrate an intra-body controllable medical device featuring magnetic field type propulsion system formed in accordance with the present invention.

FIGS. 10A, 10B and 10C illustrate an intra-body controllable medical device featuring an inflating/deflating balloon propulsion system formed in accordance with the present invention.

FIG. 11 illustrates an intra-body controllable medical device featuring gyroscopic and wing/flipper stabilization systems formed in accordance with the present invention.

FIGS. 12A-G illustrate different scope systems for deploying an intra-body controllable medical device.

FIGS. 13A, 13B and 13C illustrate an intra-body controllable medical device being deployed by a scope.

FIGS. 14A and 14B illustrate an intra-body controllable medical device being deployed into the stomach by a scope.

FIGS. 15A and 15B illustrate systems for controlling an intra-body controllable medical device.

FIGS. 16A and 16B illustrate different power supply systems for powering an intra-body controllable medical device.

FIG. 17 illustrates the use of induction charging to power an intra-body controllable medical device.

FIG. 18 illustrates tethered power transfer between two intra-body controllable medical devices.

FIG. 19 illustrates intra-device storage systems for intra-body controllable medical devices.

FIGS. 20A and 20B illustrate imaging systems that can be incorporated with an intra-body controllable medical device.

FIGS. 21A and 21B illustrates the placement of a monitoring sensor by an intra-body controllable medical device.

FIGS. 22A, 22B and 22C illustrate the delivery of a therapy system by an intra-body controllable medical device.

FIGS. 23A-D illustrate different tissue and fluid sampling devices that may be used by an intra-body controllable medical device.

FIG. 24 illustrates material dispensing systems that may be used by an intra-body controllable medical device.

FIG. 25 illustrates an interactive group of intra-body medical devices.

FIG. 26 illustrates a medical system according to embodiments described herein.

FIG. 27 illustrates a flowchart of steps undertaken in an analysis of data obtained by an intra-body controllable medical device.

FIG. 28 illustrates an intra-body controllable medical device deployed in a patient and wirelessly connected to a processing device.

FIG. 29 illustrates images taken from an intra-body controllable medical device and compared to a data set of images.

### DETAILED DESCRIPTION OF THE PREFERRED EMBODIMENT

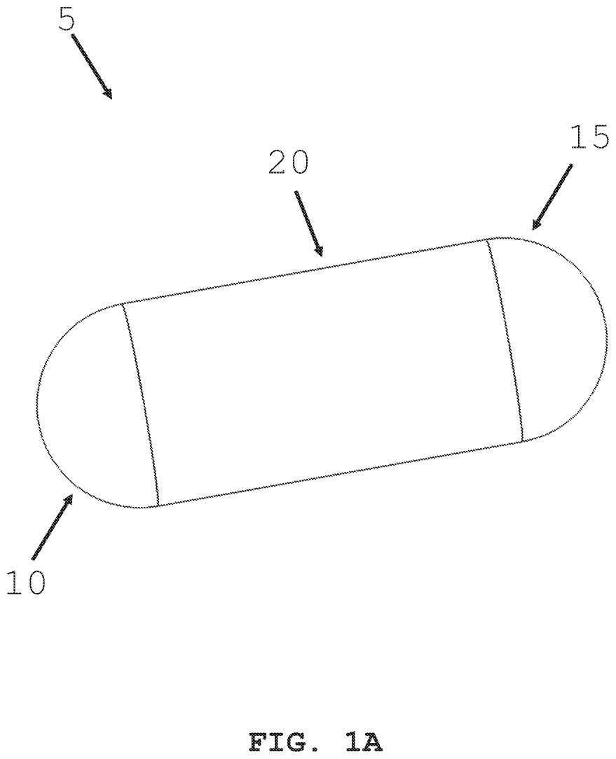

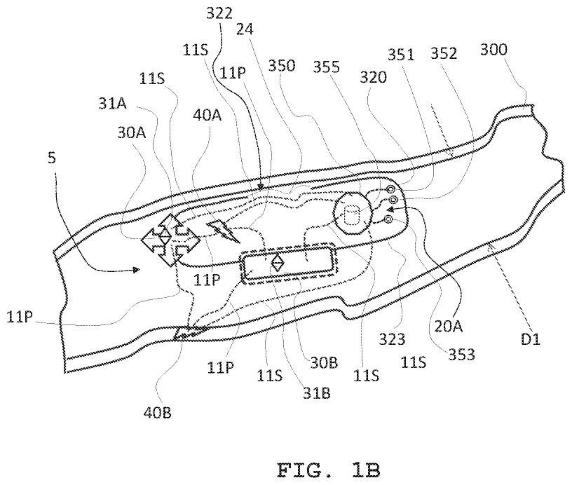

FIG. 1A illustrates an exemplary intra-body controllable medical device (hereinafter “the medical devices”). In one embodiment, the intra-body controllable medical device 5 is capsule shaped. Intra-body controllable medical device 5 has a distal end 10, a proximal end 15, and body 20 connecting the distal end 10 and proximal end 15. A control unit, a power supply system, an intra-device storage system, an imaging system, a therapy system, a sample and data gathering system, and a material dispensing system may be located within body 20 of the medical device 5, as described herein. The intra-body controllable medical device may be sized according to the anatomy that it will need to navigate, and the method used to deliver it. As an example, overall dimensions for an intra-body controllable device operating within the gastrointestinal track may have a diameter of about 25 mm and a length of about 75 mm. More preferably, the device may have a diameter of about 15 mm and a length of about 50 mm. Most preferably, the diameter may be less than about 15 mm and a length of less than about 50 mm. Overall dimensions for an intra-body controllable device that is delivered using a scope may have a diameter of about 20 mm in diameter and a length of about 75 mm. More preferably, the diameter may be about 15 mm and the length may be about 50 mm. Most preferably, the diameter may be less than 15 mm and the length less than 50 mm. Control system, power supply system, intra-device storage system, imaging system, therapy system, sample and data gathering system, and material dispensing systems are sized to fit within these dimensional guidelines.

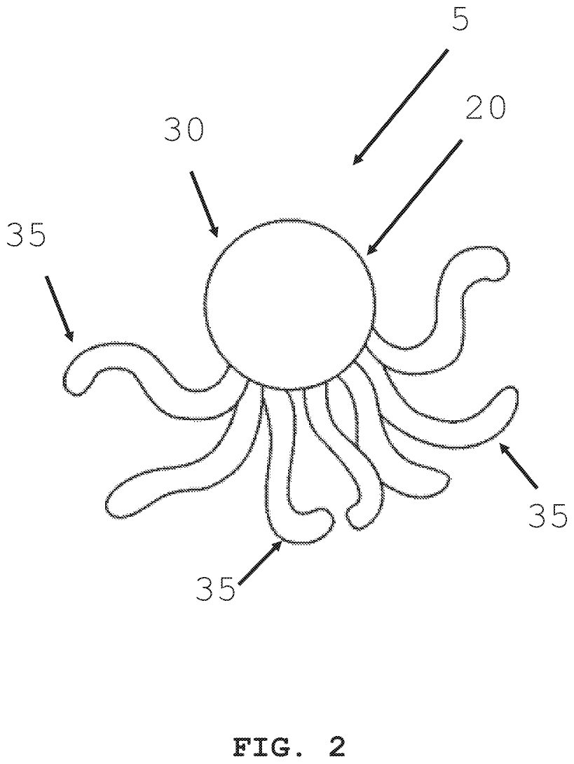

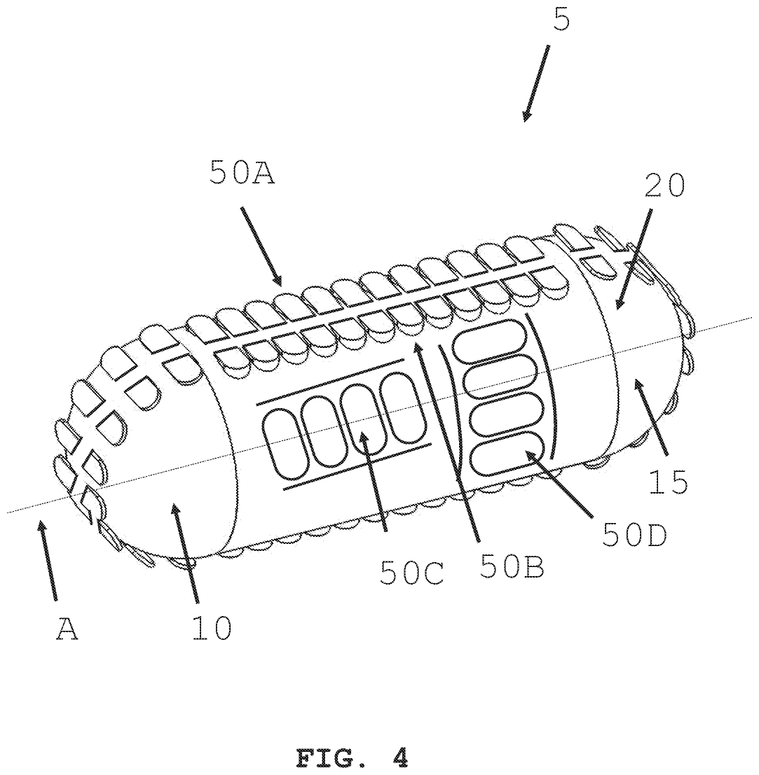

As shown in FIG. 1B, the medical device 5 includes the body 20 which is a host structure 320 that has an interior area 20A. A first propulsion system 30A and a second propulsion system 30B (e.g., a sprocket and track system similar to those shown and described with reference to FIGS. 4 and 5) are linked to the host structure 320. While the first propulsion system 30A and a second propulsion system 30B are shown and described, the present invention is not limited in this regard as only one propulsion system or more than two propulsion systems may be employed without departing from the broader aspects of the present invention. The first propulsion system 30A (e.g. see FIGS. 2-8) and the second propulsion system 30B are configurable into a peripheral boundary 323 (e.g., a skin or exterior surface) of a miniaturized size and are adapted to fit in a lumen 300 (or tissue, muscle or fat) of a living organism, such as a human. In one embodiment, the medical device 5 is configured to navigate in bone marrow within a bone. As an example, overall dimensions for an intra-body controllable device operating within the gastrointestinal track may have a diameter of about 25 mm and a length of about 75 mm. More preferably, the device may have a diameter of about 15 mm and a length of about 50mm. Most preferably, the diameter may be less than about 15 mm and a length of less than about 50 mm. Overall dimensions for an intra-body controllable device that is delivered using a scope may have a diameter of about 20 mm in diameter and a length of about 75 mm. More preferably, the diameter may be about 15 mm and the length may be about 50 mm. Most preferably, the diameter may be less than 15mm and the length less than 50 mm. In one embodiment, the host structure 320 includes an opening 322 therein for access to the interior area 20A of the host structure 320. In one embodiment, a retractable, removable or pivotable member 24 (e.g., a door, window or flap) selectively covers the opening 322. Propulsion systems 30A and 30B may be used to move device 5 within lumen 300. Additionally, propulsion systems 30A and 30B may be used to as orientation control device 31A and 31B. The propulsion systems can generate smaller and or finer movements to maintain the position of the device within the lumen 300 and can be used to change the orientation of the device within the lumen 300, tissue, muscle or fat. Controlling the orientation of the medical device 5 within the lumen 300, tissue, muscle or fat allows the intra-device storage system, imaging system, therapy system, sample and data gathering system, and/or a material dispensing system to be adjacent to a region of interest within the lumen, tissue, muscle, bone marrow or fat.

As shown in FIG. 1B, a first power supply 40A and a second power supply 40B are in communication (e.g., via power supply conductors or transmission lines or channels generally designated by the dashed lines marked 11P) with the first propulsion system 30A and the second propulsion system 30B. While the first power supply 40A and the second power supply 40B are shown and described as being in communication with the first propulsion system 30A and the second propulsion system 30B, the present invention is not limited in this regard as only one power supply or more than two power supplies may be employed and any of the power supplies (e.g., 30A or 30B) may be in communication with one or more propulsion systems (e.g., 40A or 40B).

As shown in FIG. 1B, a control unit 350 is in communication (e.g., via signal transmitting lines, wires or wireless channels, generally designated by dashed lines marked 11S) with the first propulsion system 30A, the second propulsion system 30B, the first power supply 40A and the second power supply 40B. The control unit 350 includes a computer process controller 355 that is configured to control the first propulsion system 30A, the second propulsion system 30B to move the host structure 320, the first propulsion system 30A and the second propulsion system 30B in the lumen 300 so that the host structure 320, the first propulsion system 30A, the second propulsion system 30B and the control unit 350 are self-maneuverable within the lumen 300.

As shown in FIG. 1B, a tracking device 351, a signal transmitter 352 and a signal receiver 353 are in communication with the control unit 350 via signal lines 11S for tracking and guiding the medical device 5 within the lumen 300.

As shown in the exemplary embodiment of FIG. 2, the intra-body controllable medical device 5 may be octopus shaped. The intra-body controllable medical device has a main body 30, and appendages 35. Appendages 35 may be used for propulsion, covering or wrapping the host structure 20, forming a portion of the host structure 20 or to perform a therapeutic or diagnostic task. A control unit, power supply systems, an intra-device storage system, an imaging system, a therapy system, a sample and data gathering system, and a material dispensing system similar to those shown and described with reference to FIG. 1B, may be located within main body 30 and/or appendages 35 of the device or in the interior areas 22 of the host structure 20.

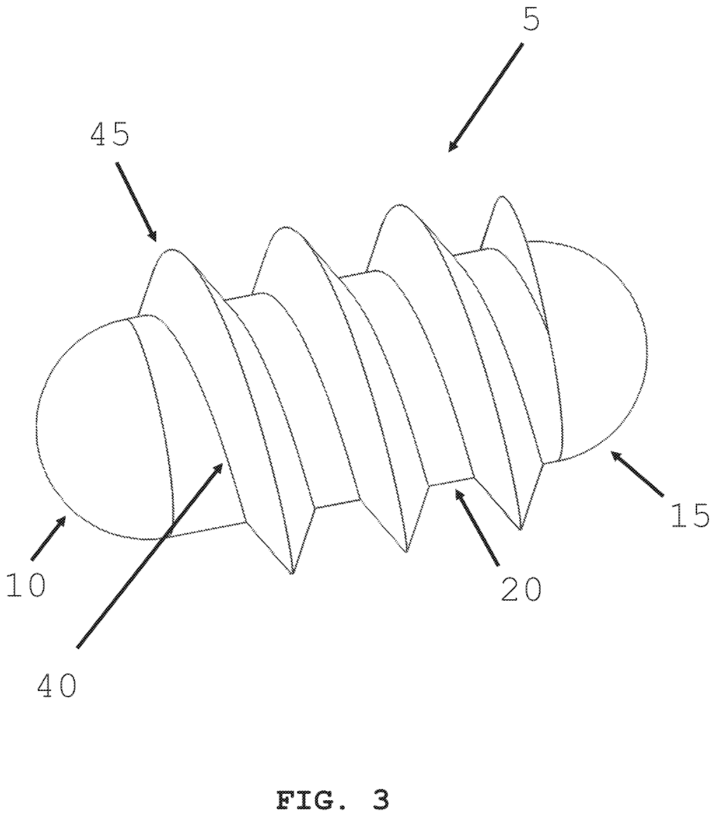

As shown in FIGS. 3-11, the present invention is generally directed to an intra-body controllable medical device and more particularly to propulsion systems for moving the intra-body controllable medical device within a lumen or orifice. The propulsion systems include one or more orientation control devices 31A, 31B per FIG. 1B, for controlling the orientation of the device within the lumen or orifice. The intra-body controllable medical device is sized to travel through lumens and/or orifices tethered and/or untethered. Thus, the intra-body controllable medical device is equipped with one or more propulsions systems, including but not limited to: (1) a sprocket driven track structure in communication with the device; (2) fluid jet stream discharging from the device; (3) an arrangement of inflating and deflating balloons in predetermined positions on and/or around the device; (4) a plurality of articulating tentacles extending from the device; (5) a screw-drive formed on external surfaces of the device and (6) stabilization wings, flippers, anchors, braces, supports and/or clamps, as described herein. The intra-body controllable medical device may also move within the body through peristalsis of the digestive system. In one embodiment, a propulsion system may be used to move device 5 to a region of interest. The device may then exit the body passively through peristalsis or may be withdrawn from the body by a tether.

Referring to FIG. 3, an intra-body controllable medical device with a screw-drive propulsion system is shown. The screw drive propulsion system has a helical thread on the external surface of the device. The helix 40 circumscribes the body 20 of the intra-body controllable medical device. A screw thread 45 is swept around the helix 40. Rotation of the helix 40 around the central axis of body 20 causes the intra-body controllable medical device to advance in the lumen 300 or orifice. Switching the direction of rotation of the helix 40 causes the intra-body controllable medical device to advance in the opposite direction.

In an alternative embodiment and referring to FIG. 4 and FIG. 5, an intra-body controllable device with a sprocket driven track structure in communication with the device is shown. The track 50 may be oriented either along the axis A of the body 20 (FIG. 4), circumferentially around body 20 (see arrow C in FIG. 5) or along one or more portions of the host structure 20 (see the second propulsion system 30B in FIG. 1B). A sprocket (not shown) may be housed within the proximal end 10 and distal end 15 of the device (FIG. 4) or central to the body 20 (FIG. 5). Movement of the track 50 relative to the body 20 of the intra-body controllable medical device 5 generates motion of the medical device.

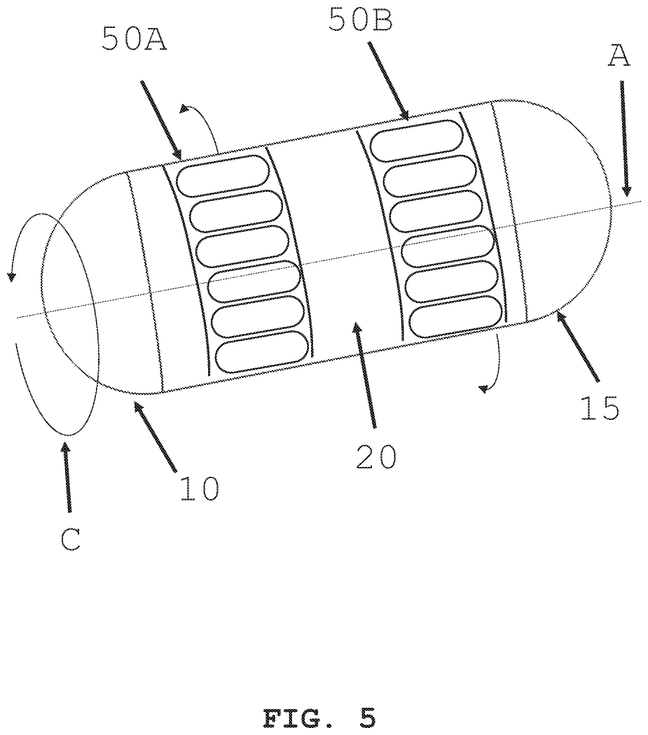

In an alternative embodiment and referring to FIG. 6, an intra-body controllable medical device with a fluid/gas jet stream discharge propulsion system is shown. The jet stream 55 of mater (e.g., gas, liquid, gel, or particles) may be released from intra-body controllable medical device 5 through a nozzle or orifice 60. The orifice 60 may be located on the distal end 10 and/or the proximal end 15 of the device. The jet stream 55 matter may be stored within body 20 of the device. Alternatively, the matter may be harvested from the body (e.g. gastric juice). Alternatively, the fluid may be harvested from the body (e.g. gastric juice) and reacted with a compound stored within device 20 (e.g. sodium bicarbonate) to create a gas (e.g. carbon dioxide gas) which can be released as a fluid/gas jet stream 55 under pressure through the orifice 60 to create propulsion. Additionally, a propeller and or turbine 61 may be located within nozzle or orifice 60. The jet stream of matter 55 may turn the turbine to generate thrust. Additionally, fluid/gas jet stream discharge propulsion system may be used as an orientation control system 31A and 31B.

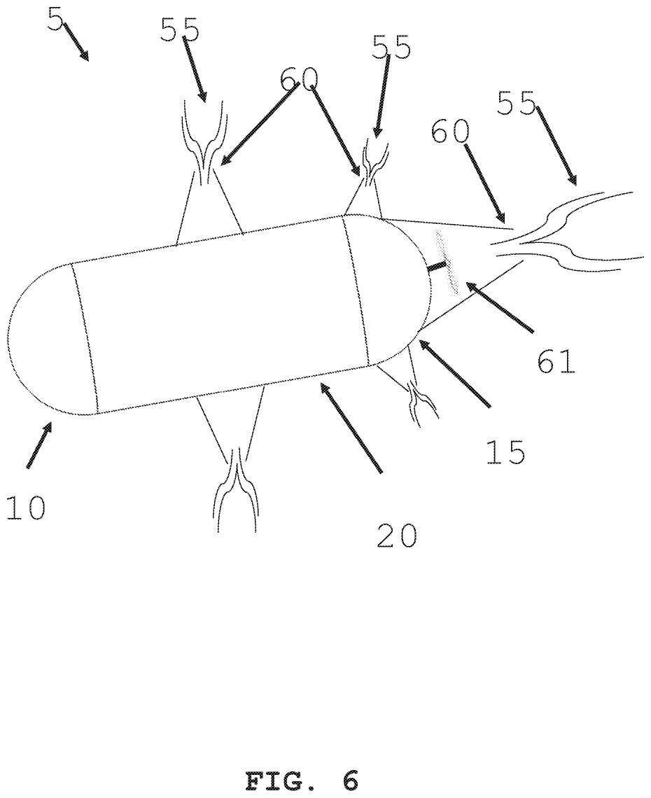

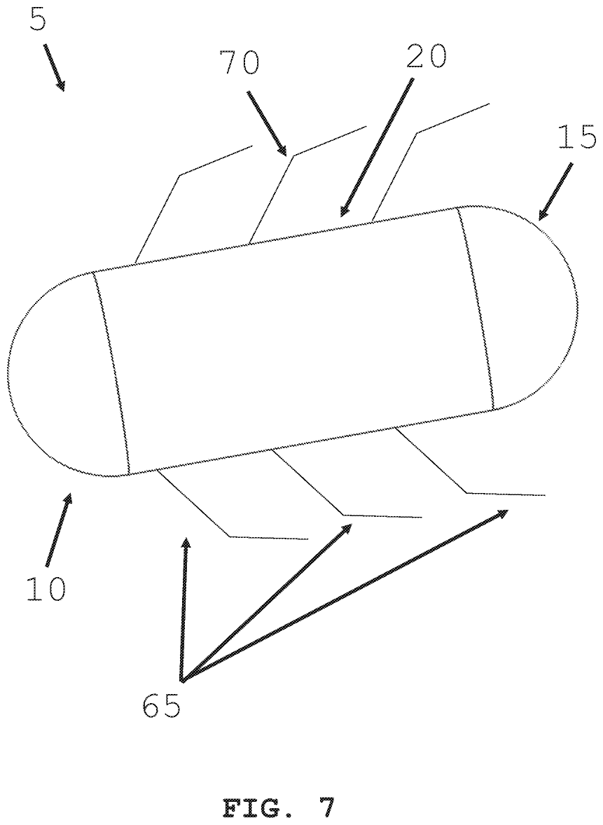

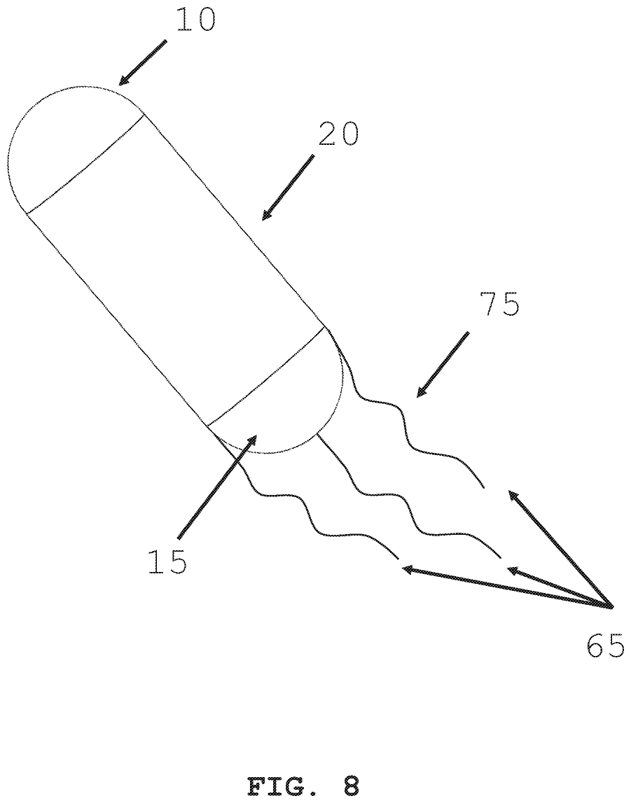

In an alternative embodiment and referring to FIG. 7 and FIG. 8, an intra-body controllable medical device 5 with a plurality of articulating tentacles extending from the body is shown. A plurality of tentacles 65 may be disposed along the length of the body 20 of the device (FIG. 7); alternatively, the tentacles 65 may be located on the distal end 10 or proximal end 15 of the device (FIG. 8). The tentacles 65 may be linear. The tentacles 65 may be linear with hinged regions 70 or may be able to articulate throughout their length 75. Motion of the tentacles 65 generates propulsion of the intra-body controllable medical device.

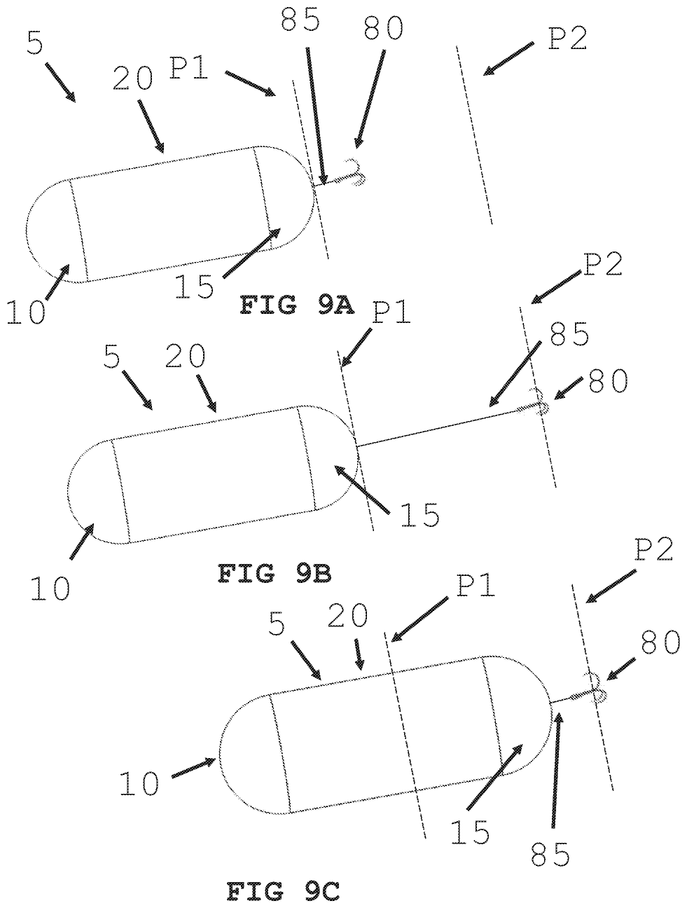

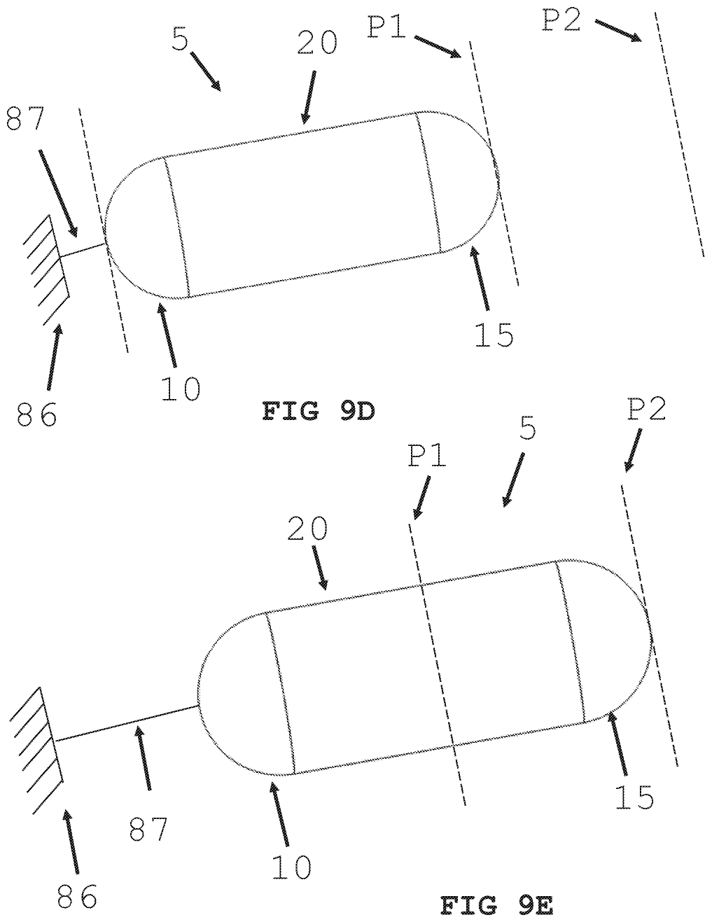

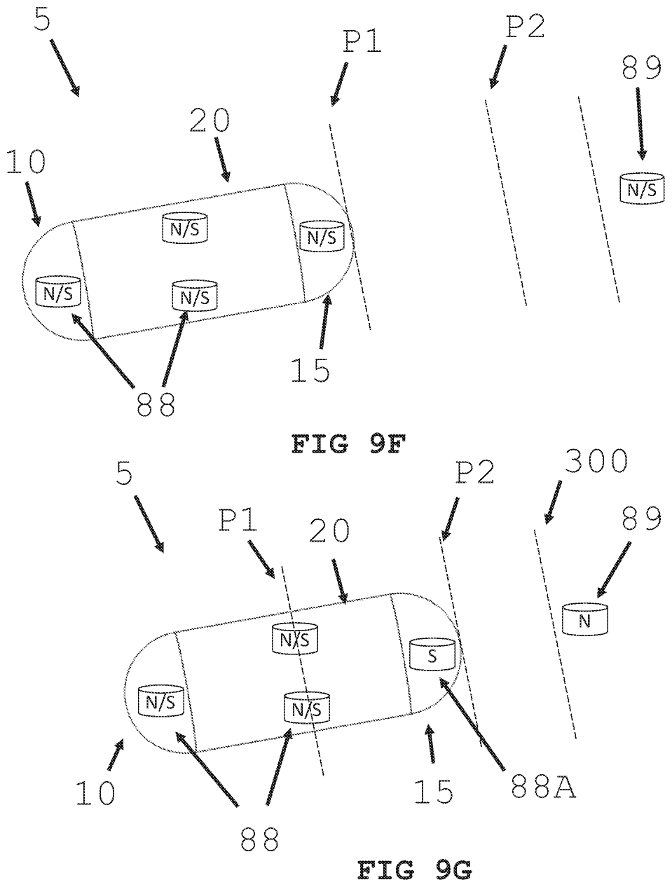

In an alternative embodiment and referring to FIGS. 9A-9G, an intra-body controllable medical device 5 with a push or pull propulsion system is shown. As shown in FIGS. 9A, 9B, and 9C, a retractable anchor-based propulsion system is shown. An anchor 80 can be any kind of anchor known in the art. As shown in FIG. 9A, the proximal end 15 is at position P1 and the anchor 80 is in the retracted position. As shown in FIG. 9B, the anchor 80 is deployed via an extended tether 85 and attaches to tissue at position P2. The anchor 80 is connected to the intra-body controllable medical device 5 by a tether 85. Propulsion is generated by retracting the tether 85 (FIG. 9C), thereby pulling the medical devices to the position P2.

In an alternative embodiment and referring to FIGS. 9D and 9E a push propulsions system is shown. As shown in FIG. 9D the proximal end 15 is at position P1 and push rod 87 is in the retracted position. The end of push rod 87 may be adjacent to a fixed structure 86. Fixed structure 86 may be lumen 300, a probe, or a scope. Propulsion is generated by advancing push rod 87 (FIG. 9E) thereby pushing the medical device to the position P2.

In an alternative embodiment and referring to FIG. 9F and FIG. 9G a push and or pull propulsion system is shown. As shown in FIG. 9F, push and or pull propulsion system utilizes magnets or magnetic fields to move device 5. Magnets may be permanent or electromagnetic. Magnets 88 are located within device 5. Additionally, there may be one or more magnets 89 located outside of lumen 300. Magnets 88 and 89 are configured to have either a north pole or a south pole. Magnets 89 may be located outside of the organism. Proximal end 15 is located at position P1. Propulsion is generated by creating an attraction force between magnet 89 and magnets 88 (FIG. 9G). An attractive force is generated between magnet 88A's south pole and magnet 89′3 north pole. This attractive force moves the medical device to position P2. Alternatively, magnet 88A's south pole (or north pole) may be aligned with magnet 89's south poll (or north pole), a repulsive force can be generated and used to push medical device 5.

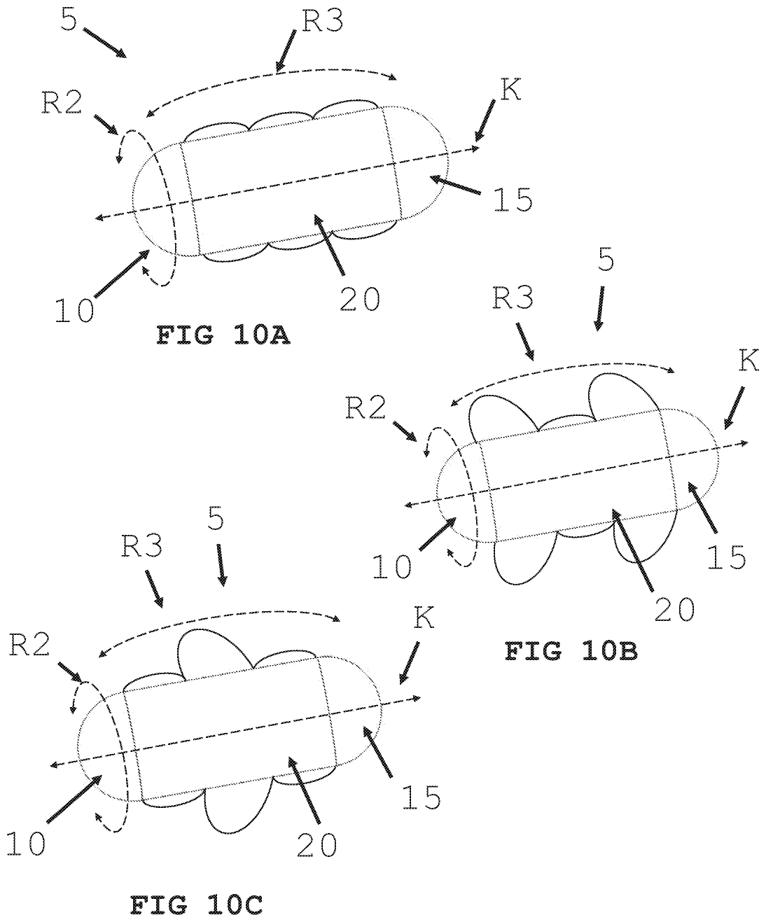

In an alternative embodiment and referring to FIG. 10, an intra-body controllable medical device 5 with an arrangement of inflating and deflating balloons 90 in predetermined positions in the direction of the arrows R and orientations (e.g., rotational or angular movement as indicated by the arrows R2 and R3) on and/or around the device is shown. The balloon 90 may be made of an elastomeric material that can be expanded under pressure yet return to its original configuration when the pressure is released. The balloon 90 may be filled with a fluid and/or a gas. When the balloon 90 is filled, the balloon increases in volume and changes shape. As an example, the balloon 90 may change to shape conformation 95 when filled with a fluid and/or gas. The fluid and/or gas may be stored within the body 20 of the medical device 5. Alternatively, the fluid may be harvested from the body (e.g. gastric juice). Alternatively, a fluid may be harvested from the body (e.g. gastric juice) and reacted with a compound stored within the device (e.g. sodium bicarbonate) to create a gas (e.g. carbon dioxide). This gas can then be used to fill and expand the balloon 90. A controller can be located within the device to direct the fluid and/or gas flow to different balloons. The rhythmic expansion and contraction of balloons can create propulsion.

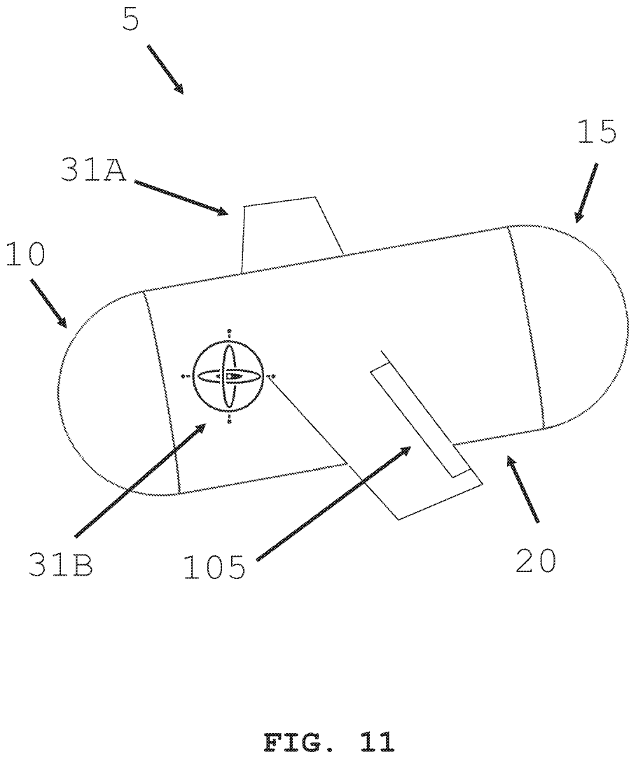

In an alternative embodiment and referring to FIGS. 1B and 11, an intra-body controllable medical device 5 may be equipped with an orientation control device (e.g. stabilization wing 31A, 31B). The orientation control device (e.g. stabilization wing 31A, 31B) is compatible with any of the propulsion systems disclosed herein. The orientation control device (e.g. stabilization wing 31A, 31B) can help guide the movement of the medical device 5 within the lumen. The orientation control device (e.g. stabilization wing 31A, 31B) may further have a flap 105 to further provide stabilization and guidance. Orientation control device may also be a gyroscope 31B. Gyroscope 31B may be used to provide stability or maintain a reference direction.

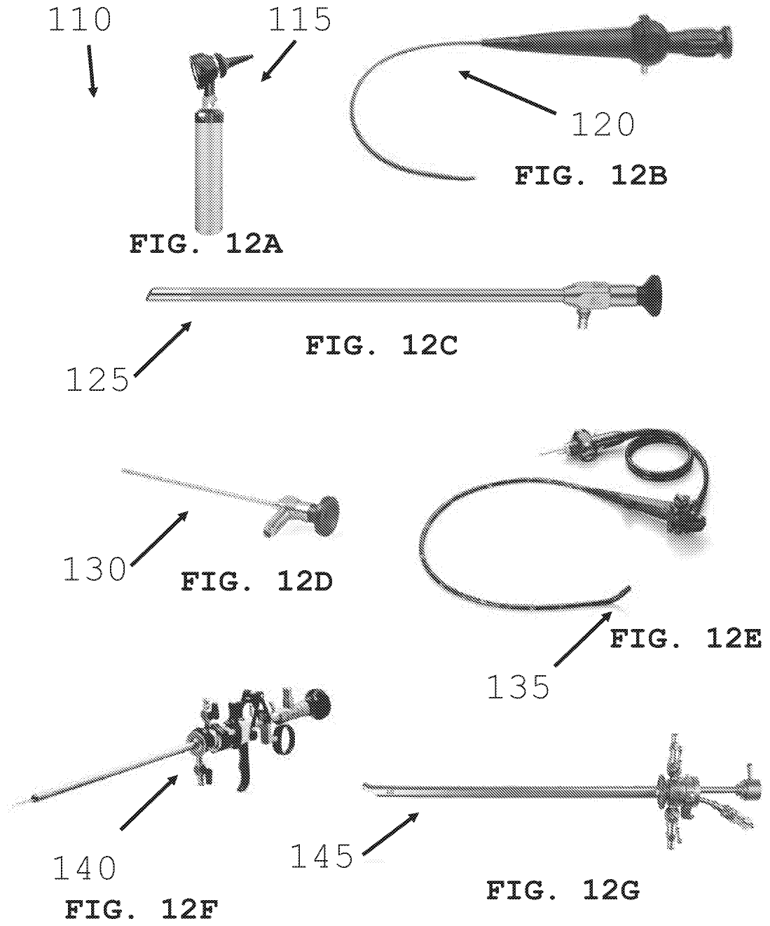

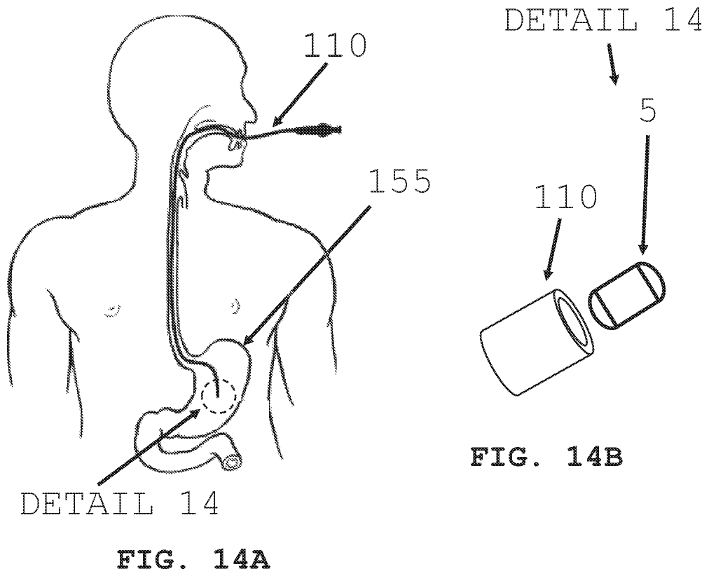

As shown in FIGS. 12A-G through FIG. 14, the present invention is generally directed to an intra-body controllable medical device 5 and more particularly to deployment devices and methods for deploying an intra-body medical device into the lumen 100. In particular, scopes for medical applications having rigid shafts or flexible conduits are configured with one or more device storage compartments, channels, actuation devices, tethers and discharge ports for deployment from one or more portions of the probe portion of the scope while positioned in a lumen. Referring to FIG. 12, the deployment device is configured to be integrated with various medical scopes 110 including but not limited to an ENT otoscope 115, a naso-pharyngoscope 120, a laparoscope 125, a sinuscope 130, a coloposcope 135, a resectoscope 145 and a cystoscope 150. Furthermore, medical device 5 may be deployed through a tube instead of a scope. Additionally, medical device 5 may be deployed via a catheter into a blood vessel or may be surgically placed (e.g. after heart surgery). Medical device 5 may be deployed though an appropriately sized needle (e.g. to gain access bone marrow) and may also be deployed generally to any area within the body (e.g. muscle, fat, and tissue) or on the skin. Medical device 5 may be deployed on the skin at the site of a wound and provide therapy (e.g. discharge clotting material like zeolite or antibacterial medication).

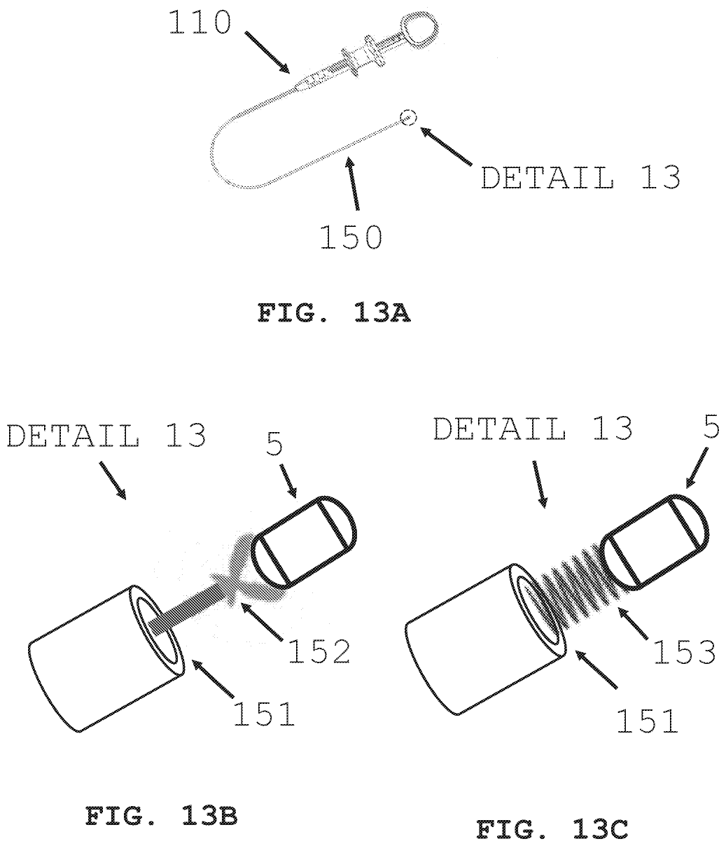

As shown in FIG. 13, the intra-body controllable medical device 5 can be deployed through the working channel 150 of the endoscope 100. The end of the working channel 150 may have a docking station 151 (FIG. 13B and FIG. 13C.) Docking station 151 may utilize a claw 152 (FIG. 13B) or a spring 153 (FIG. 13C) to hold and deploy medical device 5. Furthermore, and referring to FIG. 14A and FIG. 14B this method for deployment of the intra-body controllable medical device in the lumen 100 further includes the use of a scope 110 to deliver the device directly to the stomach 155. Alternatively, the scope 110 may be used to deliver the device directly to various organs, for example, the bladder. The method for deployment of the intra-body medical device in a lumen further includes digestion through the oral cavity, inhalation of one or more nano-sized versions of such devices for introduction to the respiratory system of a human, including the nose, pharynx, larynx, trachea, bronchi and lungs.

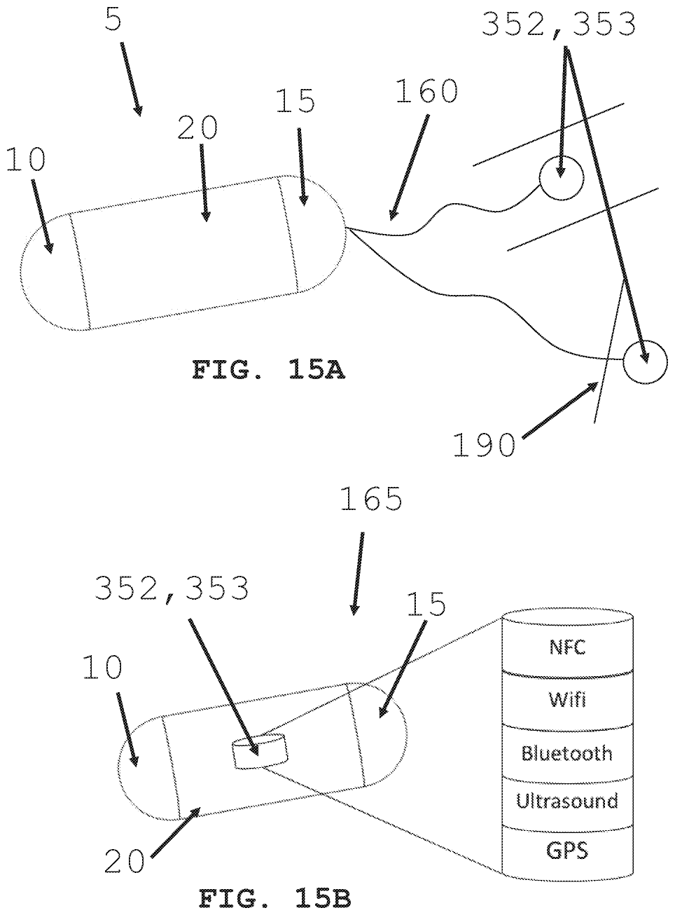

As shown in FIG. 15A and FIG. 15B, the present invention is generally directed to an intra-body controllable medical device 5 and more particularly to control and communications systems and methods for controlling and communicating with the intra-body controllable medical device in a lumen. In particular, the control and communications systems are configured to identify and track the location and orientation of the device relative to predetermined locations in the lumen and to control the device propulsion and orientation systems to guide the device to, from and around the predetermined position.

As shown in FIGS. 1B and 15A and B, the control unit 50 includes hard wired 160 (FIG. 15A) and/or wireless 165 (FIG. 15B) communication devices (e.g., transmitters 352 and receivers 353) linking an external command and monitor center with a computerized process controller 55 (FIG. 1B) in the medical device 10 which is in communication with and controls the operation of the propulsion and orientation systems based upon real time position information of the device in the body. The control unit 50 includes a software algorithm on a computer readable medium that is operable with the computerized process controller to effectuate the identification, tracking and control of the intra-body controllable medical device within the lumen.

The control unit 350 includes tracking devices 351, transmitters 352 and receivers 353, see FIG. 1B and FIG. 15 including GPS, radiation emitting sources/radiation monitoring devices, ultra sound devices, near field communication devices, Wi-Fi devices, and Bluetooth devices, that are configured to determine the position of the intra-body controllable medical device in the lumen, similar to those shown and described with reference to element numbers 315, 352 and 353 in FIG. 1B.

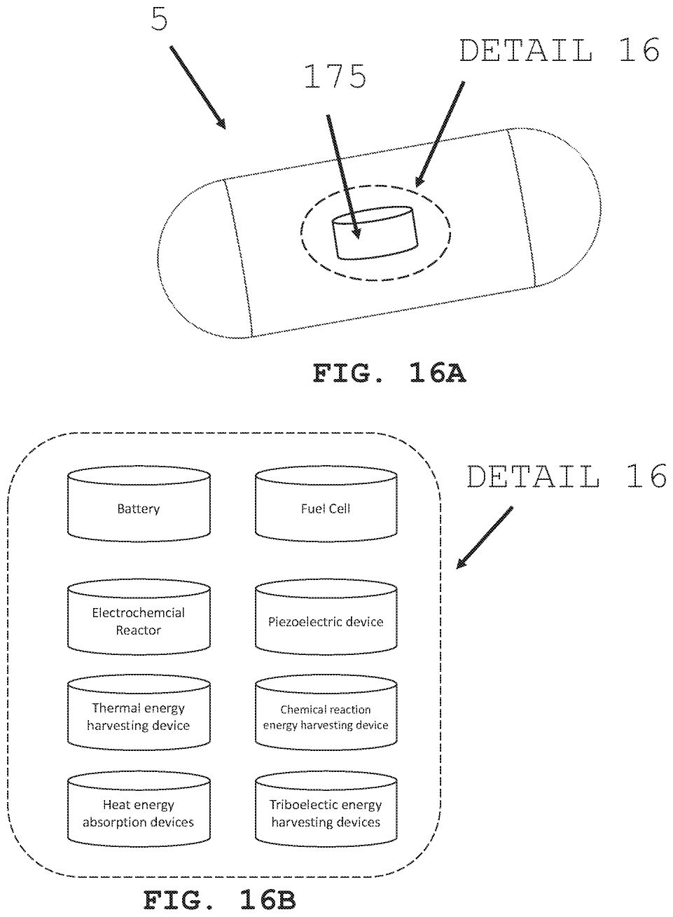

As shown in FIG. 16A and FIG. 16B, the present invention is generally directed to power supply systems 175 for an intra-body medical device and more particularly to miniaturized (e.g., computer chips having integrated circuits and positioned on integrated circuit boards [The intra-body controllable medical device may be sized according to the anatomy that it will need to navigate and the method used to deliver it. As an example, overall dimensions for an intra-body controllable device operating within the gastrointestinal track may have a diameter of about 25 mm and a length of about 75 mm. More preferably, the device may have a diameter of about 15 mm and a length of about 50 mm. Most preferably, the diameter may be less than about 15 mm and a length of less than about 50 mm. Overall dimensions for an intra-body controllable device that is delivered using a scope may have a diameter of about 20 mm in diameter and a length of about 75 mm. More preferably, the diameter may be about 15 mm and the length may be about 50 mm. Most preferably, the diameter may be less than 15 mm and the length less than 50 mm. Control system, power supply system, intra-device storage system, imaging system, therapy system, sample and data gathering system, and material dispensing systems are sized to fit within these dimensional guidelines.]) power supplies and storage devices that provide power for propulsion, control and operation of subcomponents within and around the intra-body controllable medical device and ancillary devices connectable to the intra-body controllable medical device In particular, the miniaturized power supplies include batteries, fuel cells, electrochemical reactors, piezoelectric devices, energy harvesting devices that obtain thermal and/or chemical reaction energy from the fluids in and tissue of the lumen and adjacent organs, thermal reactors heat absorption energy conversion devices and triboelectric energy harvesting devices. Batteries may include any of the kind known in the art including, but not limited to, alkaline batteries, atomic batteries, lead-acid batteries, lithium ion batteries, magnesium-ion batteries, nickel-cadmium batteries, nickel metal hydride batteries and rechargeable alkaline batteries. Electrochemical reactors may store the chemical required to create electricity within the device. Alternatively, electrochemical reactors may use fluids found within the body to react with chemicals stored within or on the device to create electricity. Piezeoelectric devices may create electricity by harvesting either the body's own motion (e.g. peristalsis) or the motion of the device as it moves within the lumen. Heat absorption devices may harvest energy from the body's temperature to create electricity. Triboelectric energy harvesting devices generate electricity as the body of the device comes into frictional contact with the lumen of the body it is passing within. Additionally, energy may be stored by the device using capacitors, thermal medium, batteries and mechanical expansion devices (e.g., springs and balloons).

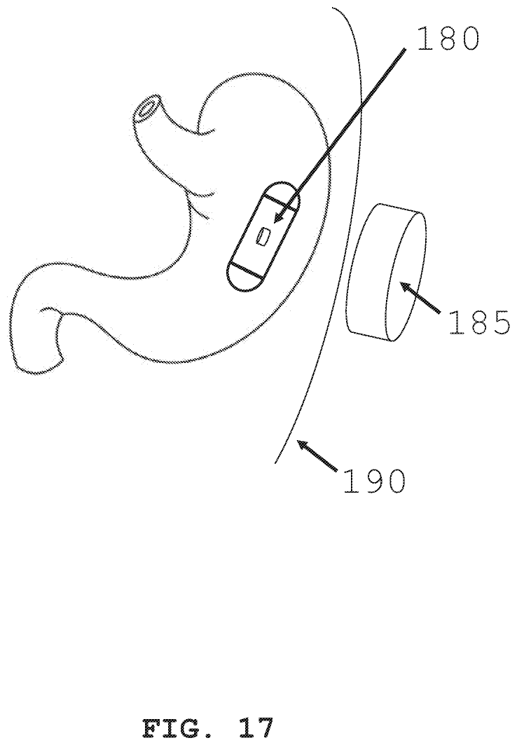

Additionally, as seen in FIG. 17, the intra-body controllable medical device 5 can be directly powered by induction energy transfer from the outside of the body 190 or inside of the body 190. An induction energy receiver 180 can be located within device 5. An induction energy transmitter 185 can be located outside body 190. Alternatively, the device may function on another internal energy storage device and be recharged by induction, charging when sufficient stored electricity has been consumed.

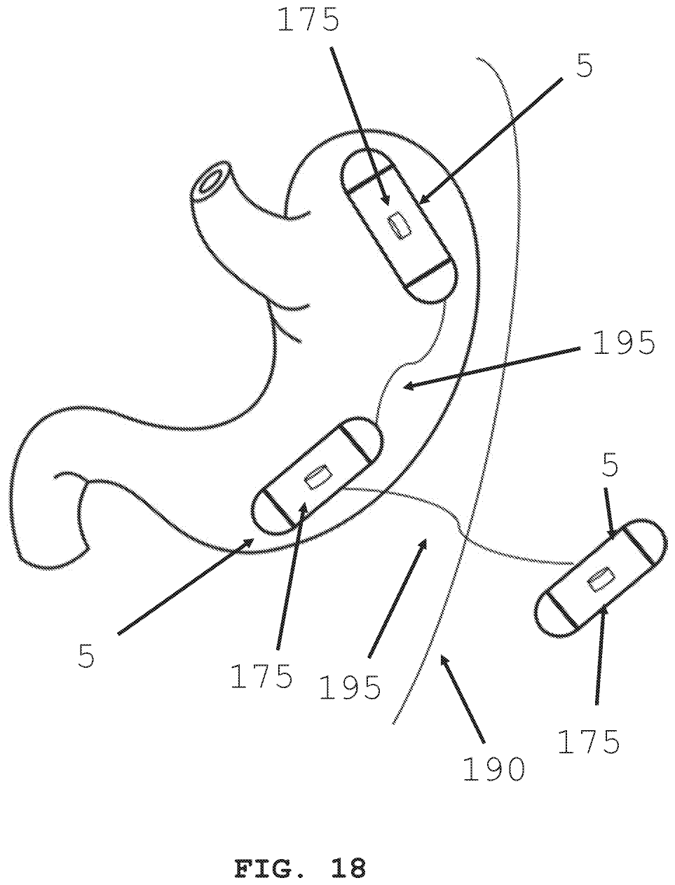

Alternatively, as seen in FIG. 18, one intra-body controllable medical device 5 can be tethered to a second intra-body controllable medical device 5. Tether 195 can transfer electricity from power source 175 in a first device to a second power source 175 of the second device. The second intra-body controllable medical device 5 may be located outside the body 190. The two devices may be permanently tethered together, or they may tether when the transfer of electricity is required.

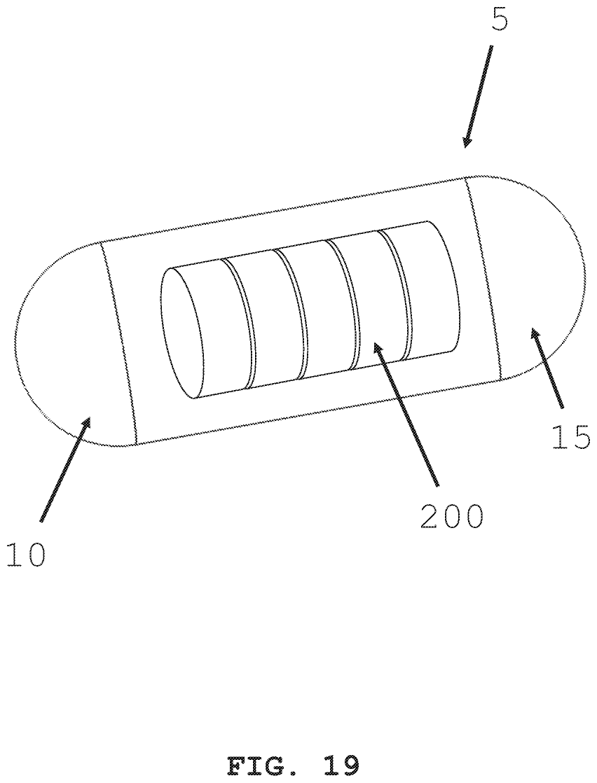

As shown in FIG. 19, the present invention is generally directed to an intra-body medical device having intra-device storage systems 200 therein and more particularly to miniaturized compartments for housing one or more power supplies, energy storage devices, medications, imaging systems, computer processor controllers, communications transmitters and receivers, propulsion systems, therapy delivering devices (e.g., radiation sources), process waste, biopsies, blood and tissue samples, medical and surgical instruments, fluids, gases, powders and consumables. The storage compartments are configured with walls, internal and external support structures, inlets, outlets, sensors (e.g., temperature, pressure and chemistry sensors), valves, pumps and ingress/egress apertures. The intra-device storage system 200 may be used to hold nerve blocking and stimulating drugs and devices, may hold devices for cleaning plaque from artery walls or may hold and deploy intestinal restrictive bands.

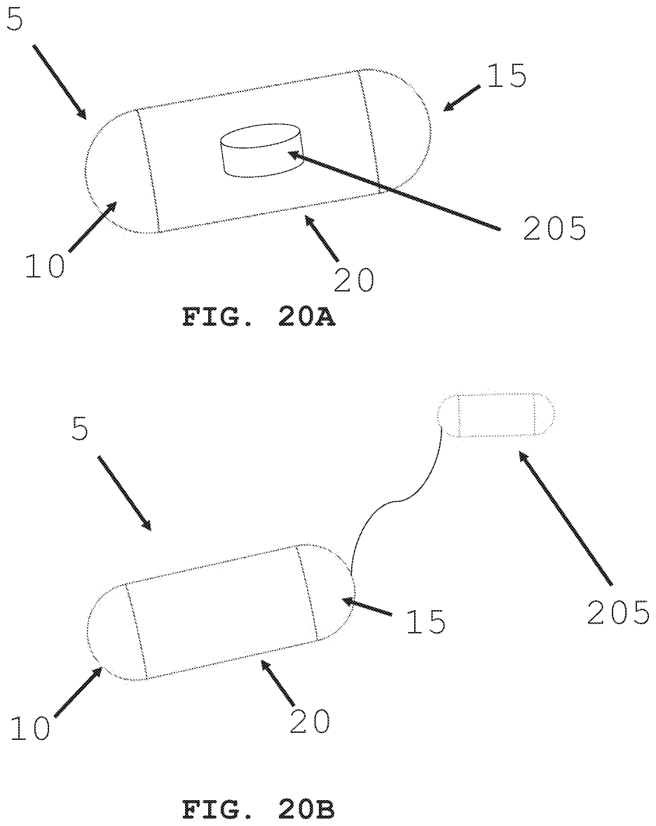

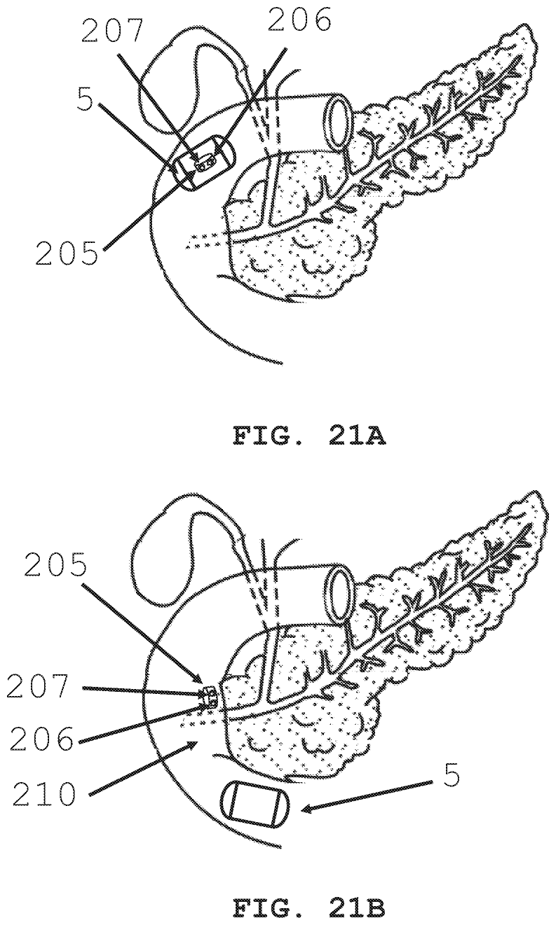

As shown in FIG. 20A, FIG. 20B and FIG. 21 the present invention is generally directed to an intra-body controllable medical device having one or more imaging systems 205 within (FIG. 20A) or remote (FIG. 20B) to the intra-body controllable medical device. The imaging systems include X-ray radiography, magnetic resonance imaging, medical ultrasonography or ultrasound, confocal microscopy, elastography, optical-coherence tomography, tactile imaging, thermography and medical digital photography. In one embodiment, the imaging systems 205 are configured to travel through the lumen 300 in the intra-body controllable medical device (FIGS. 20A and 20B). In an alternative embodiment and referring to FIG. 21A and FIG. 21B, the imaging systems 205 are further configured to be discharged from the intra-body controllable medical device while in the lumen and deposited in a predetermined location in the lumen for ongoing monitoring. As an example, and referring to FIG. 21A, the medical device 5 may be equipped with imaging system 205. The medical device 5 may travel through the small intestine and deposit imaging system 205 adjacent to the ampulla of Vater, also known as the hepatopancreatic ampulla or the hepatopancreatic duct, 210 (FIG. 21B). The medical device 5 may then continue to travel through the small intestine without the imaging system. The imaging systems 205 may be configured with a storage medium 206 to store images. The imaging systems 205 may further be configured with a transmitting device 207 to transmit real time images to one or more receivers located in other positions in the lumen and those located in other locations and organs in the body (e.g., a human body) and outside of the body.

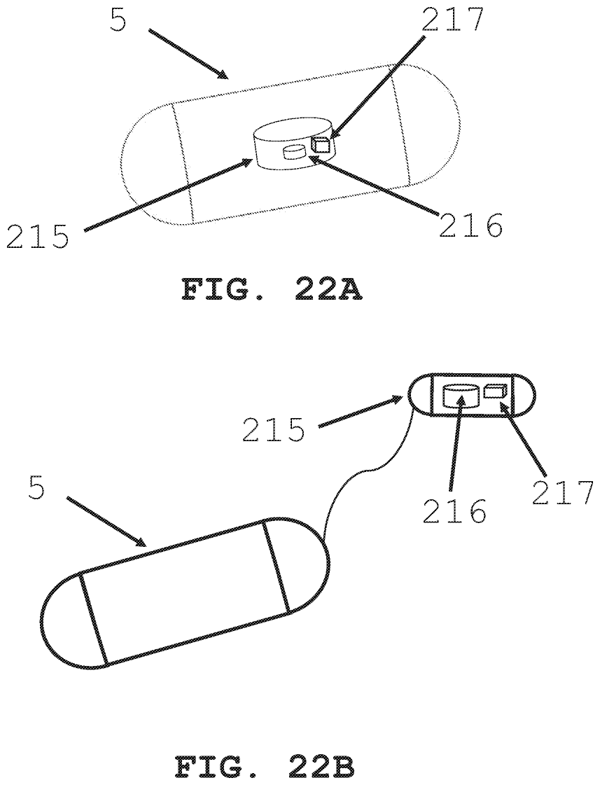

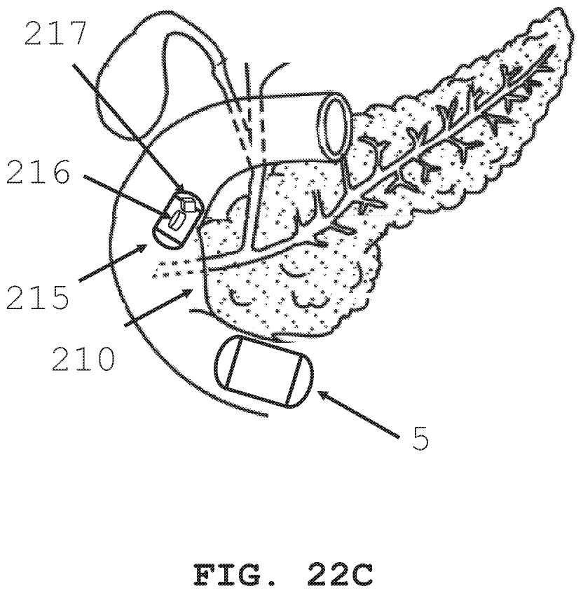

As shown in FIG. 22A, FIG. 22B, and FIG. 22C, the present invention is generally directed to an intra-body controllable medical device having one or more therapy delivery systems 215 within (FIG. 22A) or remote (FIG. 22B) to the intra-body controllable medical device. The therapy delivery systems 215 include optical-coherence tomography (OCT) guided laser instruments, radiation discharging sources, chemotherapy deploying devices, pharmaceutical and drug deploying devices, ablation devices and photodynamic therapy devices. The therapy delivery systems 215 are configured to travel through the lumen in the intra-body controllable medical device and provide therapy. The therapy delivery systems 215 may further be configured to be discharged from the device while in the lumen 100 and deposited in a predetermined location in the lumen 100 for ongoing therapy delivery (FIG. 22C). The therapy delivery systems 215 may be configured with a storage medium 216 to record time, duration and application location of the therapy. The imaging systems 205 and therapy device systems 215 may be further configured with a transmitting device 217 to transmit real time images to one or more receivers located in other positions in the lumen and those located in other locations and organs in the body (e.g., a human body) and outside of the body. The medical device 5 of FIG. 22A and FIG. 22B may travel through the small intestine and deposit therapy delivery system 215 adjacent to the ampulla of Vater, also known as the hepatopancreatic ampulla or the hepatopancreatic duct, 210 (FIG. 22C). The medical device 5 may then continue to travel through the small intestine without the imaging system.

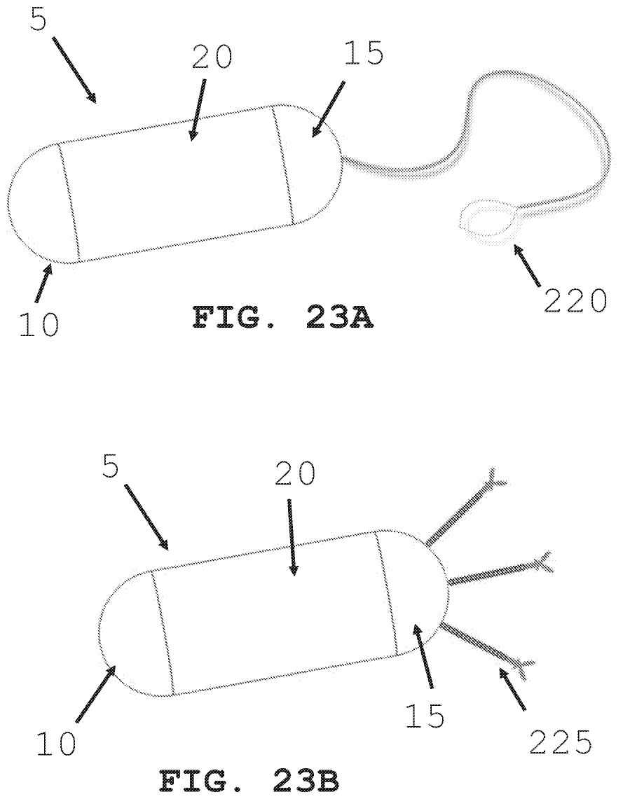

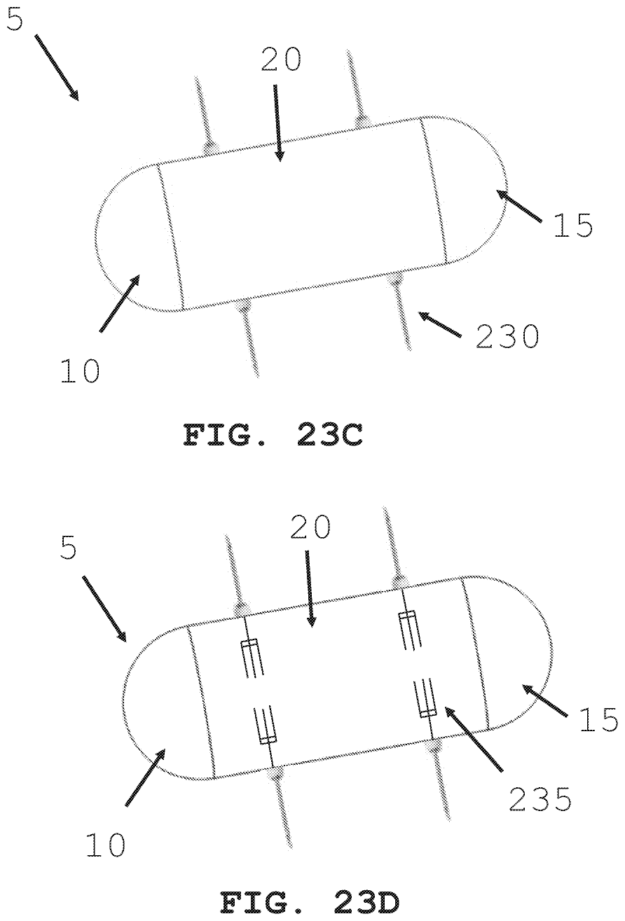

As shown in FIGS. 23A-23D, the present invention is generally directed to an intra-body controllable medical device having one or more sample and data gathering systems. The sample gathering systems are configured to obtain tissue biopsies and blood, bone, cells, bone marrow, blood, urine, DNA and fecal samples. The sample gathering devices may include any known in the art including snares 220, forceps 225, and needles 230. The data gathering devices may include pH probes, accelerometers, pressure transducers, thermometers, and dimensional measurement systems. The sample and data gathering systems are configured to perform localized testing such as complete blood counts, bone density measurements, acidity testing and turbidity testing. The sample and data gathering systems are configured to take, record and transmit dimensional, angular, velocity and volumetric measurements. The intra-body controllable medical devices contain miniaturized devices for performing the tests and obtaining the data, including miniaturized needle aspiration devices 230 (FIG. 23C), and suction devices 235 (FIG. 23D). The dimensional, angular, velocity and volumetric measurements are acquired by miniaturized devices deployed from the intra-body controllable medical device including ultrasound systems and laser imaging.

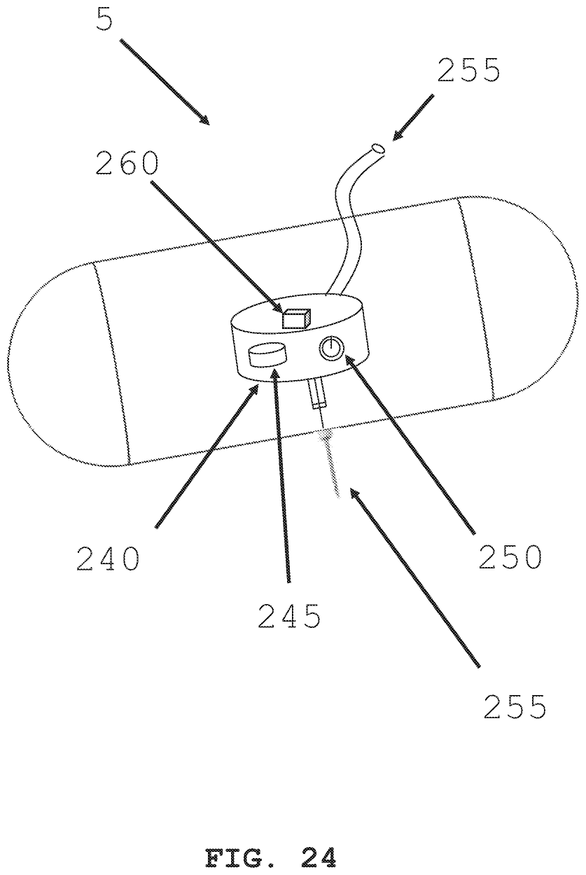

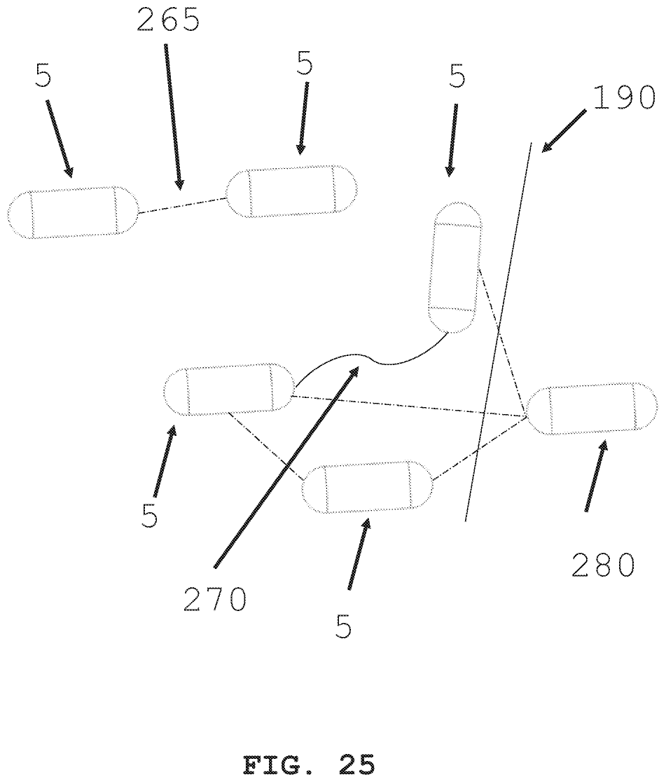

As shown in FIG. 24, the present invention is generally directed to an intra-body controllable medical device having one or more material dispensing systems. The material dispensing systems 240 are equipped with storage compartments 245 for storing and dispensing payloads including medication, liquids, powders, chemically reactive agents and radiation emitting sources and recording and tracking the location of the payloads before and after the dispensing operation. The material dispensing systems include actuators, pumps, compressors, nozzles, flow control devices 250 including valves and orifices, injection and piercing devices 255 and dose measuring and recording devices 260.

The present invention is generally directed to materials for manufacture of an intra-body controllable medical devices, and in particular to materials for such devices that are clinically inert, sterilizable, elastomeric (e.g., contractible and expandable), chemically reactive, chemically inert, dissolvable, collapsible and have physical and chemical properties to withstand exposure to bodily fluids for precise predetermined periods of time. Such materials include polymers, metallic alloys, shape memory polymers, shape memory metal alloys, shape memory ceramics, composites, silicones, thermoplastic polyurethane-based materials, excipients, zeolite adsorbents and styrene-butadiene rubbers (SBR). Materials may further include biodegradable materials such as paper, starches, biodegradable material such as gelatin or collagen.

As shown in FIG. 25, the present invention is generally directed to an interactive group of intra-body controllable medical devices. The interactive group of devices includes two or more devices 5 that are in communication with one another and/or an external computer-based control system. The two or more intra-body controllable medical devices are configured to cooperate with one another to distribute components such as power supplies, medical devices, storage compartments and auxiliary devices among the intra-body controllable medical devices so that the intra-body controllable medical devices operate together as a group to accomplish the intended functional operations and to enable the use of smaller sized individual intra-body controllable medical devices than those that would otherwise not fit into the lumen. The interactive group of intra-body controllable medical devices is configured to operate collectively as a swarm of a plurality of intra-body controllable medical devices that if deployed individually would not be as effective in undertaking the intended medical procedure or other functional operation. The interactive group of intra-body controllable medical devices includes tethering 270 or towing devices (e.g., winches) between intra-body controllable medical devices to assist in propulsion of the intra-body controllable medical devices through the lumens. Additionally, the intra-body medical devices may communicate wirelessly 265 between devices. Intra-body medical devices may communicate with a receiver or controller 280 located outside the body 190. Intra-body medical device 5 may operate like a drone, communicating and being controlled by an operator in the same room or in a different location from the patient. Furthermore, when contemplating a swarm of devices, two or more intra-body controllable medical devices 5 may be deployed. A first intra-body medical device 5 may leave the swarm group and navigate to a region of interest. This device may perform a first task and communicate back to the other devices in the swarm and direct a second device 5 to navigate to the first device 5. Second device 5 may be selected from a number of devices in the swarm because of its particular capabilities (e.g., second device 5 may have an additional battery, an imaging system, a therapy system, a sample and data gathering system, and/or a material dispensing system). Second device 5 may transfer capabilities to first device 5 or second device 5 may perform a task related to its specific capabilities. This serial communication and deployment of devices from the swarm may continue until the desired procedure is completed.

The present invention employs artificial intelligence and machine learning (hereinafter collectively referred to as “AI”) in guiding and informing the actions of the intra-body controllable medical device 5. AI is employed to enable the intra-body controllable medical device 5 to make diagnostic decisions, provide therapy, and alert the physician of a pathology.

AI is employed for reviewing the large volumes of data that the intra-body controllable medical device 5 generates. Depending on its configuration, and as described in previous figures, the device 5 may include different imaging, sensor, probe, and sample technologies. Imaging at the cellular level generates significant amounts of data, which is far too much for a physician to analyze in real-time. AI allows for the analysis of data transmitted from the device 5 and facilitates clinically relevant decisions.

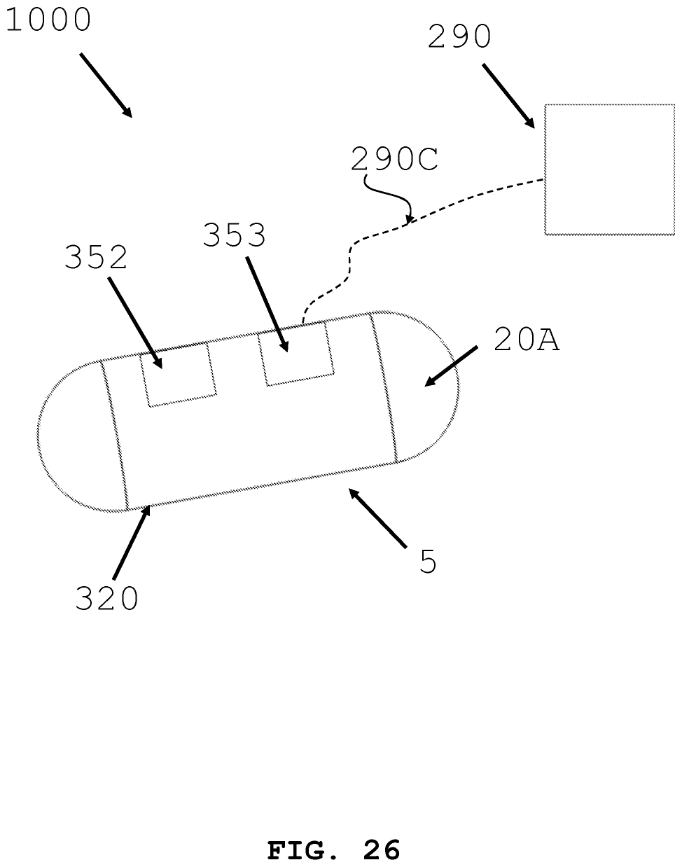

As shown in FIG. 26, the medical device 5 utilized with AI is encompassed in a medical system 1000. The medical system 1000 includes the medical device 5 for intra-body conveyance, and thus, the medical device 5 of the medical system 1000 may be deployed in a body of a patient. It is contemplated that the medical device 5 used in the medical system 1000 is of any configuration according to the embodiments described and illustrated herein, however, the medical device has a host structure 320 defining an interior area 20A. The medical device 5 of the medical system 1000 includes at least one data gathering system, such as cameras, pH probes, accelerometers, pressure transducers, thermometers, and dimensional measurement systems, and combinations of any of the foregoing. While not shown in FIG. 26, it is contemplated that the medical device 5 also includes a sample gathering system.

The medical device 5 of the medical system 1000 includes at least one means for communication, such as, for example, communication devices (e.g., transmitters 352 and receivers 353) and a processing device 290, for example a processing device that is external to the body or a processing device that is internal to the body 190 or medical device 5. The means for communication transmit data from the data gathering system to the external processing device 290 (e.g., via a communication path 290C such as a wireless communication link), which is configured to receive and analyze the transmitted data from the medical device 5. The processing device 290 implements or applies a set of instructions (also referred to as “AI instructions”, an “AI algorithm” and/or “AI technology”) that analyzes the data received from the medical device 5 and provides a user with a diagnosis and/or a recommendation for treatment.

The medical system 1000 is configured to store information and point physicians to the location of key relevant findings in a patient's body in a concise, easily discernable, and actionable format, including the use of machine learned and AI algorithms programed as software code on a computer processor located within or remote to the medical device 5. In addition, the recommendations provided by the AI instructions employed in the medical system 1000 can overlay a patient's own previous medical record to provide a more personalized treatment plan. AI technology employed in the medical system 1000 can help reduce the time between scanning, diagnosing and treating a patient. AI technology employed in the medical system 1000 can play a key role in offering faster treatment options, reducing the number of procedures a patient may undergo, and decreasing the overall financial burden on the healthcare system. AI technology employed in the medical system 1000 can reduce diagnostic and therapeutic errors that are inevitable in human clinical practice. AI technology employed in the medical system 1000 extracts useful information from larger patient population and can assist physicians and other healthcare practitioners in making real-time inferences for health risk alerts and health outcome prediction. AI technology employed in the medical system 1000 can also assist physicians by providing up-to-date medical information and best clinical practices to better inform proper patient care.

The medical system 1000 and the medical device 5 is used in a method of diagnosing or treating at least one anomaly in a patient. The method includes placing at least one medical device 5 according to any embodiment described herein into a lumen or an orifice of a patient. The medical device 5 is placed into a lumen or an orifice of a patient as described herein and as is known in the medical art. Once the medical device 5 is placed in a patient, data about the patient is collected with the medical device. The data collected by the medical device can include the data described above, which includes, but is not limited to, images, pH, size, etc. The data is collected by the sensors and data/sample gathering systems described herein. That collected data is then transmitted from the medical device 5 to an processing device 290 such as a processing device that is external to the body 190 or a processing device that is internal to the body or the medical device 5. As discussed above, the data is transmitted through devices in the medical device 5 and the processing device 290 either wirelessly or through hard wired connections. One received by the processing device 290 instructions provided to or stored on the external processing device are applied to the data received from the medical device 5. Application of the instructions analyzes the data and the analyzed data is used to diagnosis or treat the patient.

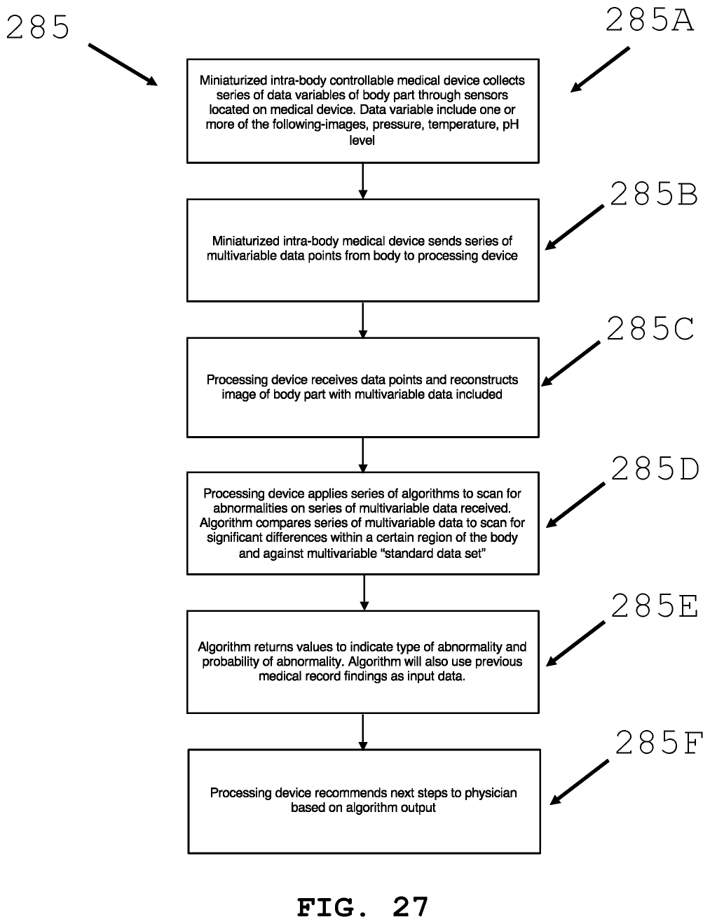

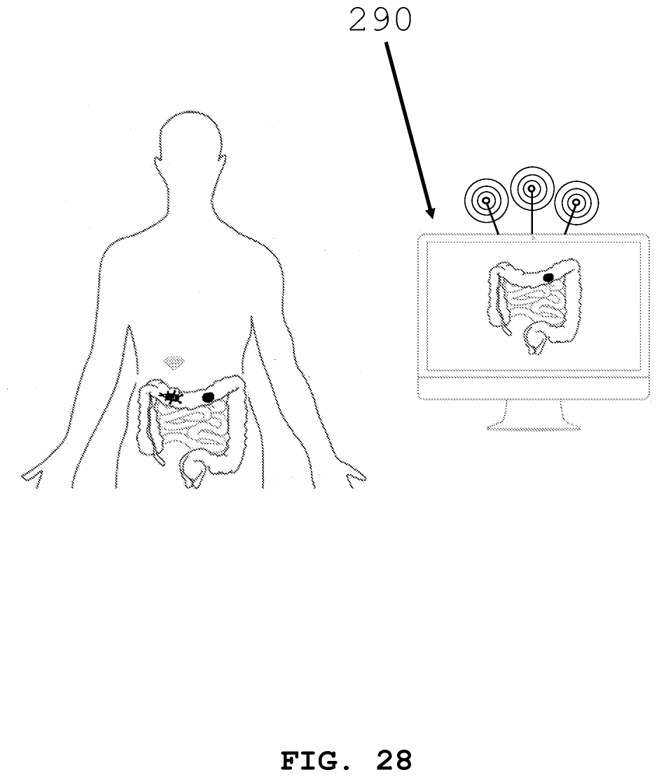

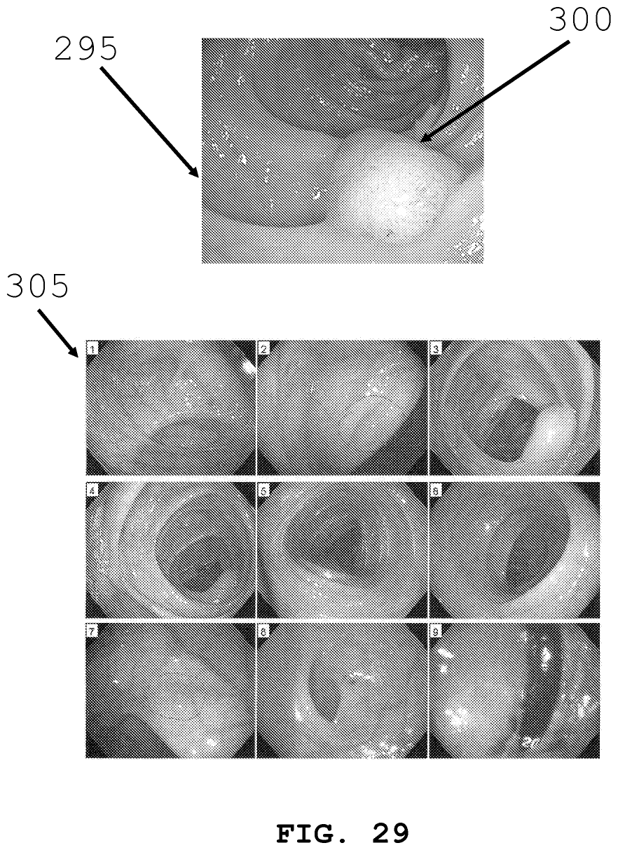

FIG. 27 shows a flow chart 285 of steps that implemented the AI algorithm for analyzing and making diagnostic and therapeutic decisions by utilizing data collected by the intra-body controllable medical device 5. As shown in FIG. 27, in step 285A, the miniaturized intra-body controllable medical device 5 collects a series of data using the data gathering system(s), e.g., sensors and imaging components. The data may include images, temperature, pH, or pressure. In real time, in step 285B, the intra-body controllable medical device transmits the data to a processing device 290 (FIGS. 26 and 28). As shown in FIGS. 26 and 28, the processing device 290 is external to the body of the patient (i.e., an external processing device). The external processing device 290 is any type of processing device configured to receive data from the medical device 5. In one embodiment, as shown by dashed lines in FIGS. 26 and 28, the external processing device 290 is in wireless communication with the medical device 5. In another embodiment, the external processing device 290 is connected to the medical device with one or more wires. The external processing device 290 is a cloud computer, a local computer terminal, or a device carried by the patient. In one embodiment, the external processing device 290 is connected to one or more graphical user interfaces for display of information to a user (e.g., physician, nurse, patient). In one embodiment, the external processing device 290 is connected to at least one of the internet, a server, and an external database. It is contemplated that more than one medical device 5 can communicate with a single external processing device 290 and that more than one external processing device 290 can communicate a single medical device 5.

Referring still to FIG. 27, in step 285C, the processing device 290 receives the data from the medical device 5, e.g., by transmission from at least one means for communication 353 and the AI algorithm stored in a computer processor disposed in the medical device, received for sources external to the medical device 5 (e.g., another medical device, the internet, a computer located outside of the body, and in step 285D, the processing device 290 compares the data received from the medical device 5 to a master data set stored on the processing device 290. The master data set contains information on both normal and pathologic samples. In steps 285E and 285F, when the data received from the medical device 5 lies outside the normal samples in the master data set, the instructions according to the AI algorithm marks the data as abnormal and compares the data to other pathologic samples in order to make a diagnosis and recommends next steps of treatment based on the application of the AI algorithm to the data received from the medical device 5 and compared to the master data set.

As seen in FIG. 29, and as an example using images, intra-body controllable medical device 5 takes an image 295 with a camera installed in or on the medical device 5. Image 295 is transmitted via means for communication 353 to the processing device 290. At processing device 290, the AI algorithm processes the image in accordance with the flow chart 285 shown in FIG. 27. Image 295 is compared to a database of other images 305. A region of interest 300 in the image 295 is identified by the AI algorithm and flagged as abnormal by the AI algorithm. The region of interest 300 is identified by the AI algorithm stored on the processing device 290 as a colonic polyp.

Depending on the diagnosis, the processing device 290 sends a signal back to the intra-body controllable medical device 5 to perform an action (i.e., a biopsy, additional imaging, etc.). Alternatively, the processing device 290 may request another intra-body controllable medical device 5 be deployed or summoned to the site of the abnormality.

The present invention is also directed to configurations for intra-body controllable medical devices and in particular to disposable, disintegrable and selectively collapsible intra-body controllable medical device s and materials and structures thereof. The intra-body controllable medical devices are manufactured of a material such as an elastomer (e.g., nitrile) that can expand and contract, for example, by inflating and deflating them. The intra-body controllable medical devices are manufactured from a biodegradable, disintegrable or dissolvable material, including paper, starches, biodegradable material such as gelatin or collagen and/or synthetic natural polymers. The collapsible intra-body controllable medical devices are configured to be flattened, extruded, stretched or disassembled in the lumen. Thus, the intra-body controllable medical devices are disposed of in the lumen or via discharge therefrom without the need to recover the intra-body controllable medical devices for analysis, inspection or future use.

The present invention is directed to methods for using intra-body controllable medical devices in the medical field and in particular for use in administering medications and therapy, deploying medical devices, imaging and surgery. The methods for using intra-body controllable medical devices includes applications in the gastro/intestinal tract (e.g. colonoscopy), urology applications, in the lungs, bladder, nasal and reproductive systems, in performing Transurethral Resection of Bladder Tumors (TURBT), Transurethral Resection of the Prostate (TURP) and transrectal prostate ultrasound, biopsy, and radiation treatment. The methods for using intra-body controllable medical devices include use in procedural environments, operatory/surgical procedures, ambulatory/out-patient procedures and unobtrusive normal routine living.

Although the present invention has been disclosed and described with reference to certain embodiments thereof, it should be noted that other variations and modifications may be made, and it is intended that the following claims cover the variations and modifications within the true scope of the invention.

1. A medical device for intra-body conveyance, the medical device comprising:
a host structure defining an interior area (20A);
at least one propulsion system linked to the host structure;
the host structure and the at least one propulsion system being configurable into a peripheral boundary of a size adapted to fit in a lumen of a living organism;
at least one power supply in communication with the at least one propulsion system;
a control unit in communication with the at least one propulsion system and the power supply, the control unit having a computer process controller configured to control the at least one propulsion system to move the host structure and the at least one propulsion system in the lumen so that the host structure and the at least one propulsion system are self-maneuverable within the lumen.

2. The medical device of claim 1, wherein the propulsion system comprises at least one of:
a sprocket driven track structure in communication with the host structure;
a fluid jet stream discharging from the host structure;
a plurality of articulating tentacles extending from the host structure;
a screw-drive formed on external surfaces of the host structure;
at least one of a pull device and a push device in communication with the host structure; and
an arrangement of inflating and deflating balloons, the balloons being at least one of:
in predetermined positions on the host structure, and
in predetermined positions around the host structure.

3. The medical device of claim 1, wherein the at least one propulsion system comprises an orientation control device configured for orientation control of the medical device within the lumen.

4. The medical device of claim 3, wherein at least one of the orientation control devices and the at least one propulsion system comprises at least one of stabilization wings, flippers, anchors, braces, supports, clamps, and a gyroscope, ballast systems.

5. The medical device of claim 1, further comprising a docking station for receiving at least one of a tether, a medical scope and a second medical device.

6. The medical device of claim 5, wherein the medical scope is at least one of an ENT otoscope, a naso-pharyngoscope, a laparoscope, a sinuscope, a coloposcope, a resectoscope and a cystoscope.

7. The medical device of claim 5, wherein the docking station includes at least one of the tether, a holding device, a release device, a launch device, a push device and a pull device.

8. The medical device of claim 1, further comprising at least one of a tracking device, a signal transmitter and a signal receiver in communication with the control unit for tracking and guiding the medical device within the lumen.

9. The medical device of claim 1, wherein the at least one power supply comprises at least one of a miniaturized batteries, fuel cell, electrochemical reactor, piezoelectric device, energy harvesting device that obtains thermal and/or chemical reaction energy from the fluids in and tissue of the lumen and adjacent organs, thermal reactors, heat absorption energy conversion devices and triboelectric energy harvesting devices.

10. The medical device of claim 1, wherein the host structure comprises at least one storage system comprising miniaturized compartments for housing one or more power supplies, energy storage devices, medications, imaging systems, computer processor controllers, communications transmitters and receivers, propulsion systems, therapy delivering devices (e.g., radiation sources), process waste, biopsies, blood and tissue samples, medical and surgical instruments, fluids, gases, powders and consumables.

11. The medical device of claim 1, wherein the host structure comprises at least one of a clinically inert material, a sterilizable material, an elastomeric material, a chemically reactive material, a chemically inert material, a disintegrable material, a dissolvable material, a collapsible material and a material having physical and chemical properties to withstand exposure to bodily fluids for predetermined periods of time.

12. The medical device of claim 1, wherein the host structure comprises at least one imaging system, the at least one imaging system being selected from the group consisting of X-ray radiography, magnetic resonance imaging, medical ultrasonography or ultrasound, confocal microscopy, elastography, optical-coherence tomography, tactile imaging, thermography and medical digital photography.

13. The medical device of claim 12, wherein the at least one imaging system is configured to travel through the lumen in the medical device.

14. The medical device of claim 12, wherein the at least one imaging system is configured to be discharged from the medical device while in the lumen and deposited in a predetermined location in the lumen for ongoing monitoring.

15. The medical device of claim 1, wherein the host structure comprises at least one therapy delivery system, the at least one therapy delivery system selected from the group consisting of optical-coherence tomography (OCT) guided laser instruments, radiation discharging sources, chemotherapy deploying devices, pharmaceutical and drug deploying devices, and photodynamic therapy devices.

16. The medical device of claim 15, wherein the at least one therapy delivery system is configured to travel through the lumen in the medical device and provide therapy.

17. The medical device of claim 15, wherein the at least one therapy delivery system is configured to be discharged from the medical device while in the lumen and deposited in a predetermined location in the lumen for ongoing therapy delivery.

18. The medical device of claim 15, wherein the at least one therapy delivery system is configured with a storage medium configured to record at least one of record time, duration and application location of the therapy.

19. The medical device of claim 1, wherein the host structure comprises at least one of a sample gathering system and a data gathering system.

20. The medical device of claim 19, wherein the sample gathering system is configured to obtain at least one of tissue biopsies and blood, bone, cells, bone marrow, blood, urine, DNA and fecal samples.

21. The medical device of claim 19, wherein the data gathering devices comprise at least one of pH probes, accelerometers, pressure transducers, thermometers, and dimensional measurement systems.

22. The medical device of claim 1, wherein the host structure comprises at least one material dispensing system equipped with at least one storage compartment configured for at least one of storing and dispensing payloads, the payloads comprising at least one of medication, liquids, powders, chemically reactive agents and radiation emitting sources.

23. The medical device of claim 22, wherein the at least one material dispensing system comprises at least one of an actuator, a pump, a compressor, a nozzle, a flow control device, an injection device, a piercing device a dose measuring device and a recording device.

24. An interactive group of at least two of medical devices according to claim 1, wherein the interactive group of the at least two medical devices are in communication with at least one of an external computer-based control system and each other and are configured to cooperate with each another to perform at least one predetermined mission.

25. A method for using a medical device according to claim 1, the method being directed to at least one of administering medications, administering therapy, deploying medical devices, imaging and surgery.

26. A method for using a medical device according to claim 1, the method being directed to at least one of use in a gastro/intestinal tract, use in urology applications, use in a lung, use in a bladder, use in a nasal system, use in a reproductive system, use in performing Transurethral Resection of Bladder Tumors (TURBT), use in Transurethral Resection of the Prostate (TURP), use in trans rectal prostate ultrasound, biopsy, and radiation treatment.

27. A method for using a medical device according to claim 1, the method being directed to use in procedural environments, operatory and surgical procedures, ambulatory and out-patient procedures and unobtrusive normal routine living.

28. A plurality of medical devices according to claim 1 in communication with at least one repository (555), the repository comprising at least one of a heat sink, a chemical reactor and a storage vessel, at least one of the plurality of medical devices comprising at least one of a cooling system and a material discharge system, wherein the at least one repository is positioned in at least one of intra body and outside the body.

29. A medical system (1000) comprising a medical device according to claim 1 configured to perform data analysis on the data received from the medical device, the data analysis comprising at least one of machine learning and artificial intelligence.

30. A medical system comprising:
at least one medical device for intra-body conveyance, the medical device comprising:
a host structure defining an interior area; and
at least one data gathering system; and
at least one communication device for transmitting data from the at least one data gathering system, the medical device being configurable into a peripheral boundary of a size adapted to fit in a lumen of a living organism;
an external processing device configured to receive the transmitted data from the at least one communication device and configured to perform data analysis on the data received from the medical device.

31. The medical system according to claim 30, wherein the data from the at least one data gathering system is selected from a group consisting of images, pH values, temperatures, positions, forces, pressures, dimensions, time, and combinations of the foregoing.

32. The medical system according to claim 31 wherein the images are obtained by one of white light, contrast enhancement using dye, optical methods, electronic methods, narrow band imaging, auto fluorescence, confocal laser microscopy, optical coherence tomography, fluorescence, reflectance spectroscopy, targeted imaging, and multimodal imaging.

33. The medical system according to claim 30, wherein the external processing device is selected from the group consisting of a local computer terminal, a cloud computer terminal, and a portable device.

34. The medical system according to claim 30, wherein the data analysis performed by the external processing device comprises:
review of the data for unusual patterns and anomalies;
application of instructions to the data to compare data received from the medical device to data stored in a database in communication with the external processing device.

35. The medical system according to claim 34, wherein the review of the data for unusual patterns and anomalies comprises analysis of at least one of: gross shape; morphology;
cell shape; size; nuclei shape; size and number of nuclei; structure of chromatin; scattering properties; pH level, temperature level; pressure; and tactile level.

36. The medical system according to claim 30, wherein the data analysis provides a probability of abnormality including those of neoplastic lesions, ulcers and polyps.

37. The medical system according to claim 30, wherein the data analysis provides recommendations for next steps.

38. The medical system according to claim 37, wherein the next step can be performed by the medical device.

39. A method of diagnosing or treating at least one anomaly in a patient, the method comprising:
placing at least one medical device according to claim 1 into a lumen or an orifice of a patient;
collecting data about the patient with the medical device;
transmitting the data about the patient from the medical device to an external processing device;
applying instructions to the data received from the medical device to analyze the data; and
utilizing the data analysis to diagnosis or treat the patient.

40. The method according to claim 39, wherein the data is selected from a group consisting of images, pH values, temperatures, positions, forces, pressures, dimensions, time, and combinations of the foregoing.

41. The method according to claim 39, wherein the images are obtained by one of white light, contrast enhancement using dye, optical methods, electronic methods, narrow band imaging, auto fluorescence, confocal laser microscopy, optical coherence tomography, fluorescence, reflectance spectroscopy, targeted imaging, and multimodal imaging.

42. The method according to claim 39, wherein the external processing device is selected from the group consisting of a local computer terminal, a cloud computer terminal, and a portable device.

43. The method according to claim 39, wherein the data analysis performed by the external processing device comprises:
reviewing the data for unusual patterns and anomalies; and
applying instructions to the data to compare data received from the medical device to data stored in a database in communication with the external processing device.

44. The method according to claim 43, wherein the review of the data for unusual patterns and anomalies comprises analysis of at least one of: gross shape; morphology; cell shape; size; nuclei shape; size and number of nuclei; structure of chromatin; scattering properties; pH level, temperature level; pressure; and tactile level.

45. The method according to claim 39, wherein the data analysis provides a probability of abnormality including those of neoplastic lesions, ulcers and polyps.

46. The method according to claim 39, wherein the data analysis provides recommendations for next steps.

47. The method according to claim 46, wherein the next step can be performed by the medical device.
